(19) United States
(12) Patent Application Publication
(10) Pub. No.: US 2022/0074758 A1
(43) Pub. Date: Mar. 10, 2022

(54) METHOD, APPARATUS, AND SYSTEM FOR PROVIDING LANE-LEVEL ROUTING OR MAPPING BASED ON VEHICLE OR USER HEALTH STATUS DATA

(71) Applicant: HERE Global B.V., Eindhoven (NL)

(72) Inventor: Priyank SAMEER, Mumbai (IN)

(21) Appl. No.: 17/095,525

(22) Filed: Nov. 11, 2020

Related U.S. Application Data
(60) Provisional application No. 63/076,166, filed on Sep. 9, 2020.

(51) Int. Cl. G01C 21/36; G16H 10/60; G06N 20/00; G07C 5/08; G08G 1/16

(52) U.S. Cl. CPC G01C 21/3658; G16H 10/60; G08G 1/166; G07C 5/0808; G07C 5/085; G06N 20/00

(57) ABSTRACT

An approach is provided for providing lane-level mapping/routing based on vehicle/user health status data. The approach, for example, involves determining health status data indicating at least one health or maintenance condition of a vehicle, a user of the vehicle, or a combination thereof. The approach also involves computing a lane-level navigation routing for the vehicle, another vehicle, or a combination thereof based on the health status data. The approach further involves providing the lane-level navigation routing as an output.

### RELATED APPLICATION

This application claims priority from U.S. Provisional Application Ser. No. 63/076,166, entitled “METHOD, APPARATUS, AND SYSTEM FOR PROVIDING LANE-LEVEL ROUTING OR MAPPING BASED ON VEHICLE OR USER HEALTH STATUS DATA,” filed on Sep. 9, 2020, the contents of which are hereby incorporated herein in their entirety by this reference.

### BACKGROUND

Navigation and mapping service providers are continually challenged to provide users up-to-date data on traffic flow and congestion. Vehicle mechanical failures account for 13% of traffic accidents, and around one-third of traffic accidents are caused by driver negligence, such as speeding, falling asleep, distracted driving, recklessness, or inexperience. To improve the safety of highly automated driving or autonomous driving, the navigation and mapping service providers see a potential of incorporating vehicle sensor data in traffic data to reduce crashes and improve safety. The existing traffic reporting systems offered by vehicle manufacturers usually use vehicle sensors to monitor traffic, then alert a driver or an autonomous vehicle to operate safely. Such systems merely serve road traffic information.

### SOME EXAMPLE EMBODIMENTS

Therefore, there is a need for providing lane-level mapping/routing based on vehicle/user health status data determined based on vehicle service date data, distances driven since a service visit, user sleep time data, user work time data, user health check-up dates, user current health conditions, etc.

According to one embodiment, a method comprises determining health status data indicating at least one health or maintenance condition of a vehicle, a user of the vehicle, or a combination thereof. The method also comprises computing a lane-level navigation routing for the vehicle, another vehicle, or a combination thereof based on the health status data. The method further comprises providing the lane-level navigation routing as an output.

According to another embodiment, an apparatus comprises at least one processor, and at least one memory including computer program code for one or more computer programs, the at least one memory and the computer program code configured to, with the at least one processor, cause, at least in part, the apparatus to determine health status data indicating at least one health or maintenance condition of a vehicle, a user of the vehicle, or a combination thereof. The apparatus is also caused to compute a lane-level navigation routing for the vehicle, another vehicle, or a combination thereof based on the health status data. The apparatus is further caused to provide the lane-level navigation routing as an output.

According to another embodiment, a computer-readable storage medium carries one or more sequences of one or more instructions which, when executed by one or more processors, cause, at least in part, an apparatus to determine health status data indicating at least one health or maintenance condition of a vehicle, a user of the vehicle, or a combination thereof. The apparatus is also caused to compute a lane-level navigation routing for the vehicle, another vehicle, or a combination thereof based on the health status data. The apparatus is further caused to provide the lane-level navigation routing as an output.

According to another embodiment, an apparatus comprises means for determining health status data indicating at least one health or maintenance condition of a vehicle, a user of the vehicle, or a combination thereof. The apparatus also comprises means for computing a lane-level navigation routing for the vehicle, another vehicle, or a combination thereof based on the health status data. The apparatus further comprises means for providing the lane-level navigation routing as an output.

In addition, for various example embodiments of the invention, the following is applicable: a method comprising facilitating a processing of and/or processing (1) data and/or (2) information and/or (3) at least one signal, the (1) data and/or (2) information and/or (3) at least one signal based, at least in part, on (or derived at least in part from) any one or any combination of methods (or processes) disclosed in this application as relevant to any embodiment of the invention.

For various example embodiments of the invention, the following is also applicable: a method comprising facilitating access to at least one interface configured to allow access to at least one service, the at least one service configured to perform any one or any combination of network or service provider methods (or processes) disclosed in this application.

For various example embodiments of the invention, the following is also applicable: a method comprising facilitating creating and/or facilitating modifying (1) at least one device user interface element and/or (2) at least one device user interface functionality, the (1) at least one device user interface element and/or (2) at least one device user interface functionality based, at least in part, on data and/or information resulting from one or any combination of methods or processes disclosed in this application as relevant to any embodiment of the invention, and/or at least one signal resulting from one or any combination of methods (or processes) disclosed in this application as relevant to any embodiment of the invention.

For various example embodiments of the invention, the following is also applicable: a method comprising creating and/or modifying (1) at least one device user interface element and/or (2) at least one device user interface functionality, the (1) at least one device user interface element and/or (2) at least one device user interface functionality based at least in part on data and/or information resulting from one or any combination of methods (or processes) disclosed in this application as relevant to any embodiment of the invention, and/or at least one signal resulting from one or any combination of methods (or processes) disclosed in this application as relevant to any embodiment of the invention.

In various example embodiments, the methods (or processes) can be accomplished on the service provider side or on the mobile device side or in any shared way between service provider and mobile device with actions being performed on both sides.

For various example embodiments, the following is applicable: An apparatus comprising means for performing a method of the claims.

Still other aspects, features, and advantages of the invention are readily apparent from the following detailed description, simply by illustrating a number of particular embodiments and implementations, including the best mode contemplated for carrying out the invention. The invention is also capable of other and different embodiments, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

The embodiments of the invention are illustrated by way of example, and not by way of limitation, in the figures of the accompanying drawings:

FIG. 1 is a diagram of a system capable of providing lane-level mapping/routing based on vehicle/user health status data, according to one embodiment;

FIGS. 2A-2C are diagrams of example lane-level mapping/routing events, according to one embodiment;

FIG. 3 is a diagram of components of a traffic platform capable of providing lane-level mapping/routing based on vehicle/user health status data, according to one embodiment;

FIG. 4 is a flowchart of a process for providing lane-level mapping/routing based on vehicle/user health status data, according to one embodiment;

FIG. 5 is a diagram of a cloud-base vehicle/user heath or maintenance data infrastructure, according to one embodiment;

FIG. 6 is a diagram of an example user interface for setting a safety risk score display level, according to various embodiments;

FIGS. 7A-7B are diagrams of example user interfaces for presenting safety risk scores at different display levels in a routing mode, according to various embodiments;

FIG. 8 is a diagram of a geographic database, according to one embodiment;

FIG. 9 is a diagram of hardware that can be used to implement an embodiment;

FIG. 10 is a diagram of a chip set that can be used to implement an embodiment; and

FIG. 11 is a diagram of a mobile terminal (e.g., handset or vehicle or part thereof) that can be used to implement an embodiment.

### DESCRIPTION OF SOME EMBODIMENTS

Examples of a method, apparatus, and computer program for providing lane-level mapping/routing based on vehicle/user health status data are disclosed. In the following description, for the purposes of explanation, numerous specific details are set forth in order to provide a thorough understanding of the embodiments of the invention. It is apparent, however, to one skilled in the art that the embodiments of the invention may be practiced without these specific details or with an equivalent arrangement. In other instances, well-known structures and devices are shown in block diagram form in order to avoid unnecessarily obscuring the embodiments of the invention.

As used herein, the term “vehicle health status data” indicates at least one health or maintenance condition of a vehicle that can be determined based on, for example, a vehicle service date, a distance driven by the vehicle since a service visit, etc. As used herein, the term “user health status data” indicates at least one health or maintenance condition of a user that can be determined based on, for example, a user sleep time, a user work time, a health check-up date of the user, a current health condition of the user, etc. Although various embodiments are described with respect to manually driven vehicles, it is contemplated that the approach described herein may be used with autonomous vehicles. The term “autonomous vehicle” is often used interchangeably with self-driving vehicles, driverless car and/or robot car.

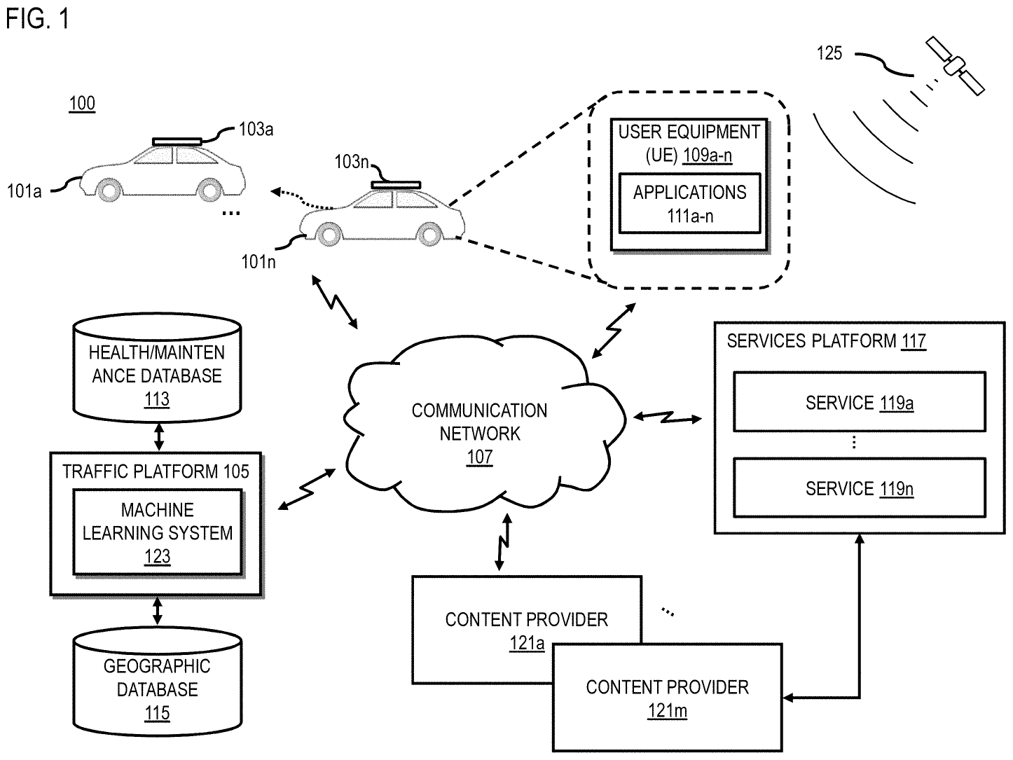

FIG. 1 is a diagram of a system capable of providing lane-level mapping/routing based on vehicle/user health status data, according to one embodiment. Automated driving has been a hot trend in recent years and is quickly becoming a reality following advances in machine learning, computer vision, and compute power. Generally, an autonomous vehicle is a vehicle driving on the road without human intervention. An autonomous vehicle uses different sensor technologies (e.g., a camera sensor, Light Detection and Ranging (LiDAR), etc.) and a high-definition (HD) map or dynamic backend content including traffic information services to travel on a road network with little or no human intervention.

As mentioned, around one-third of accidents are caused by driver negligence, such as speeding, falling asleep, distracted driving, recklessness, or inexperience, while 13% of accidents are resulted from mechanical failure, which can be from normal wear and tear or inadequate service or vehicle maintenance. Currently, there are vehicle software and sensors monitoring vehicle's health conditions (e.g., tire pressures, fuel levels, etc.) and/or driver's health conditions (e.g. blood pressure, eye gaze, etc.). The user or the vehicle may appear normal based on the sensor data, while having underlining conditions that post safety risks but undetectable via the sensor data. For example, the driver is lack of sleep but appears energized after consuming energy drinks. As another example, the vehicle has not yet visited a dealership to resolve recall issues that can trigger sudden failures of engines, brakes, seatbelts, signal lights, etc.

In addition, providing users of manually driving, autonomous or semi-autonomous vehicles (e.g., drivers or passengers) with up-to-date data on traffic flow and lane-level routing can reduce crashes with vehicles of poor health status and improve safety on the road network. Safe autonomous operations will be significantly improved if high health risk vehicles/drivers can be determined and shared through traffic channels and/or an interconnected transport infrastructure that support autonomous driving. For example, it is critical to be aware of the high health risk vehicles/drivers, so that navigation systems can generate safe lane-level routing of the current vehicle (e.g., routing it away from the high health risk vehicles/drivers) and/or safe lane-level routing of the high health risk vehicles/drivers (e.g., routing them off the road and/or the road network).

To address these problems, the system 100 of FIG. 1 introduces a capability to provide lane-level mapping/routing based on vehicle/user health status data and to generate relevant alerts of high health risk vehicles/drivers to warn current user(s) and/or upstreaming users (e.g., of autonomous vehicles, highly assisted driving (HAD) vehicles, semi-autonomous vehicles, or manually driving vehicles) via traffic channels, e.g., multiple traffic message channels (TMCs), vehicle-to-vehicle (V2V) communication services, vehicle-to-everything (V2X) communication services, etc.

In one embodiment, the system 100 can collect and process vehicle health/condition data (such as when was the last servicing done, total kms/miles driven/last garage visit based on location history data), drivers health condition data (e.g., sleeping time data, working time data, last health check-up data, current health condition data, etc.), to share health data via a digital map and/or the traffic channels, thus increasing safety for vehicles travelling on the road network.

For instances, vehicle accidents can be caused by drivers who are suffering from a pre-existing medical condition, some of which can make it unsafe for a person to operate a vehicle such as heart diseases, poor eyesight, diabetes, seizure disorders, etc. In another instance, driver fatigue increases the risk of crashes by up to a third. Driver fatigue is preventable, but still a road safety problem.

In one embodiment, the system 100 can analyze vehicle/user health or maintenance data directly as collected. In one embodiment, the system 100 can extract the vehicle/user health or maintenance data from one or more digital channels (e.g., smart home, smart phones, smart speakers, etc.), the digital map and/or the traffic channels, in order to predict the vehicle/user health status data and generate lane-level vehicle mapping/routing.

By way of example, the system 100 can detect a high health risk vehicle, i.e., whether a vehicle or its driver is likely in a poor health status (i.e., a sleepy driver), by analyzing vehicle/user health data, vehicle probe data, along with map data. The vehicle probe data may include timestamp data, geolocation data, speed data, . . . etc. The system 100 then alerts one or more upstream vehicles to take one or more strategies to avoid the high health risk vehicle, such as changing to different lane, exiting the road, using a service road, etc. The system 100 can alert the high health risk drivers (e.g., the driver is sick or tired after 15-hr shift) to let autonomous vehicle to take over, or alert drivers of high health risk vehicles (e.g., likely to breakdown) to swap vehicles if possible.

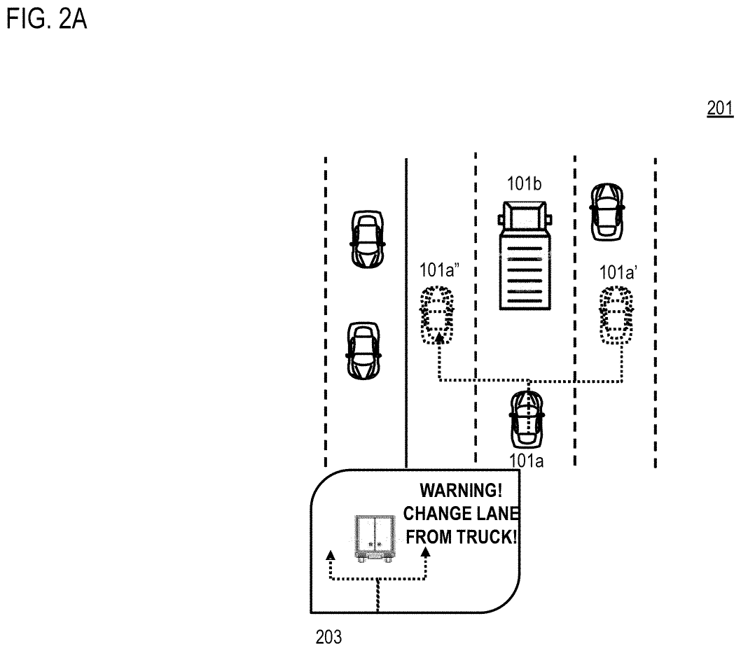

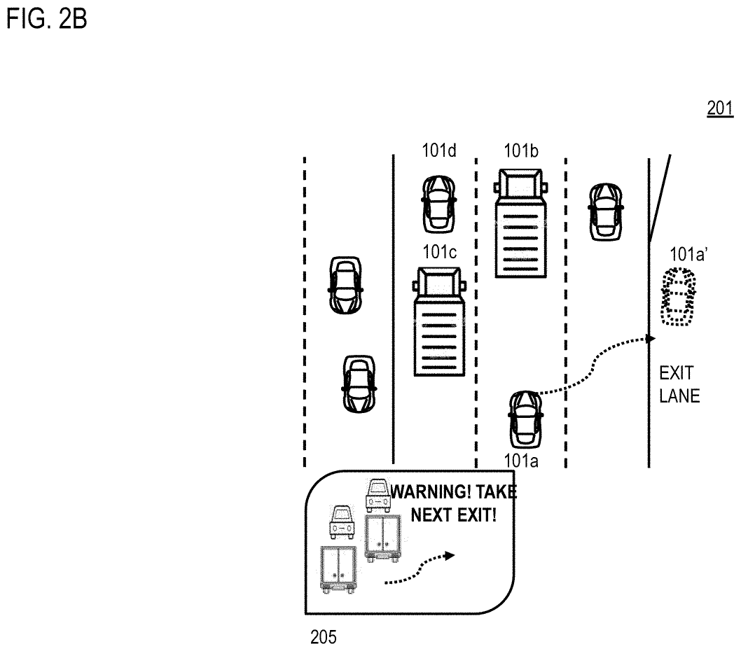

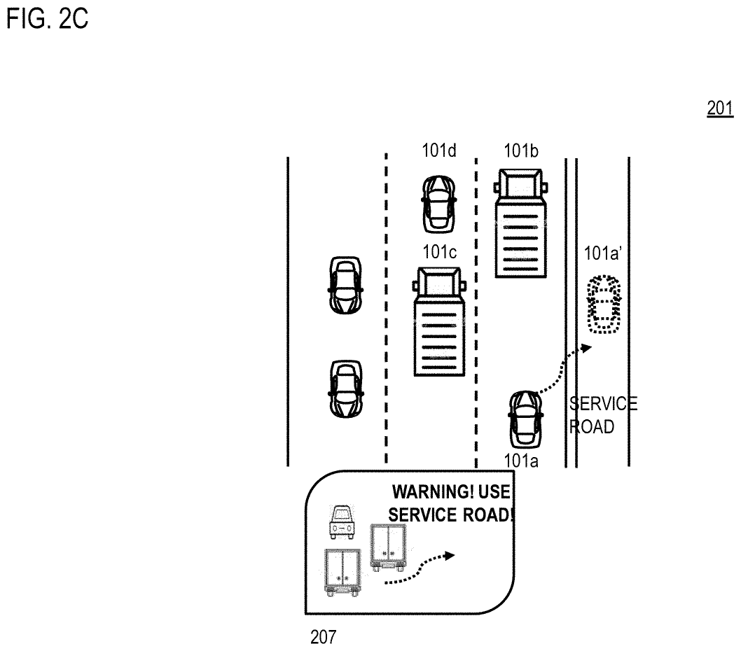

FIGS. 2A-2C are diagrams of example lane-level mapping/routing events, according to various embodiments. In FIG. 2A, a vehicle 101a is moving in the middle lane of a vehicle path 201, when the system 100 determines a high health risk vehicle (e.g., a truck 101b) in front of the vehicle 101a. The system 100 can send an alert 203 to vehicle 101a to change to the right lane (e.g., to a position shown as a dot-lined vehicle 101a′) or the left lane (e.g., to a position shown as a dot-lined vehicle 101a″) of the vehicle path 201, to avoid the truck 101b. A lane normal switching or overtaking event by the vehicle 101a may be a three-step procedure: perception, decision, and vehicle control. So, the vehicle moving path may cross the lane smoothly at a departure angle α and the vehicle speed did not change dramatically. The alert 203 includes an instruction: “Warning! Change lane from truck!” and a diagram of the back of the truck and two possible moving lines.

In FIG. 2B, the vehicle 101a is moving in the middle lane of the vehicle path 201, when the system 100 determines a plurality of high health risk vehicles (e.g., trucks 101b, 101c, vehicle 101d, etc.) in front of the vehicle 101a. The system 100 can send an alert 205 to vehicle 101a to exit the vehicle path 201 via a ramp, to avoid the high health risk vehicles. The alert 205 includes an instruction: “Warning! Take next exit!” and a diagram of the backs of the high health risk vehicles and a moving line.

In FIG. 2C, the vehicle 101a is moving in the right lane of the vehicle path 201, when the system 100 determines a plurality of high health risk vehicles (e.g., trucks 101b, 101c, vehicle 101d, etc.) in front of the vehicle 101a. The system 100 can send an alert 207 to vehicle 101a to exit the vehicle path 201 to a service road parallel with the path 201, to avoid the high health risk vehicles. The alert 207 includes an instruction: “Warning! Use service road!” and a diagram of the backs of the high health risk vehicles and a moving line.

Although FIGS. 2A-2C show the movements by the vehicle 101a, it is contemplated that the approaches described herein may be used for the movement(s) by the high health risk vehicle(s). By way of example, the system 100 may suggest all high health risk vehicles moving to a dedicated lane (e.g., a slow-moving-vehicle lane, a most right lane, a shoulder, etc.). A high health risk vehicle can easily get out of a road from the most right lane, e.g., to pull over to the emergency shoulder, to the nearest hospital, etc.

As another example, the system 100 can look at map data for a wide shoulder on the left for a high health risk driver to rest or for a vehicle swap. Sometimes, the middle lane is chosen when both left and right lanes have aggressive traffic/drivers. As another instance, when a high health risk vehicle if getting off the highway after a few exits, the system 100 can move the high health risk vehicle to the right lane earlier (although the right lane is slow), since the driver/vehicle is impaired and cannot switch lanes at a normal speed. The system 100 can control the optimal timing of serving an alert. For example, the same maneuver of 100-meter in advance of the position for a normal vehicle/driver will be advanced to 200-meter to give more time for the slow/bad vehicle/driver to make the move.

Although various embodiments are described with respect to avoid high health risk vehicles/drivers, it is contemplated that the approach described herein may be used with other cases to encounter high health risk vehicles/drivers or the like. For instance, a tow truck wants to go to lanes, road segments, roads, areas, etc. where more cars are predicted to break down via a vehicle health data approach. By way of example, the road segment has over 90% of the vehicles serviced within the last 3 months versus another road segment has only 25% of the vehicles serviced within 3-6 months, and yet another road segment has 60% of the vehicles serviced more than one year ago. In this example, the system 100 will suggest the tow truck to the last road segment (poor vehicle maintenance) to wait for vehicles to break down. A similar approach can be applied for the police to go to a higher crime area (e.g., drug traffic), for emergency responders to go to an area caught with more fire, etc.

In short, the system 100 can collect (or use) vehicle health status and driver/user health condition information, to generate a lane level routing. The system 100 can also use historical health status data to predict (or provide) current vehicle health status data and driver health status data. The system 100 can further applying the health status data to map building context, such as generating a health status map data layer based on map-matched health status data to layover a map. The system 100 can store lanes (or routes) information and the health status data in a map and used for other purposes (such as lane-level routing etc.). The system 100 can provide the health status data via the map and/or traffic channels to users to be aware of nearby high health risk vehicles/users and to avoid traffic accidents.

The Society of Automotive Engineers International defines driving automation are six levels: Level 0 (automated system has no sustained vehicle control), Level 1 (“hands on”), Level 2 (“hands off”), Level 3 (“eyes off”), Level 4 (“mind off”), and Level 5 (“steering wheel optional”). The system 100 can improve dynamic traffic content delivery on HAD in an open location platform pipeline (OLP) for Level 3 or above autonomous driving. The system 100 can improve driver and/or vehicle awareness of high health risk vehicles/drivers on the road network via the health status data for all levels 0-5 in a vehicle-to-everything (V2X) communication scheme and big data environment, etc.

Safe autonomous operations generally require the map data to provide at least a lane-level granularity (e.g., so that navigation systems can avoid lane departure crashes). In one embodiment, the system 100 collects a plurality of instances of probe data and/or vehicle sensor data (e.g., on-board diagnostics, OBD) from one or more vehicles 101a-101n (also collectively referred to as vehicles 101) (e.g., autonomous vehicles, HAD vehicles, semi-autonomous vehicles, etc.) having one or more vehicle sensors 103a-103n (also collectively referred to as vehicle sensors 103) (e.g., LiDAR, global positioning system (GPS), camera sensor, etc.) and having connectivity to the traffic platform 105 via the communication network 107. In one instance, probe data may be reported as probe points, which are individual data records collected at a point in time that records telemetry data for that point in time. A probe point can include attributes such as: (1) probe ID, (2) longitude, (3) latitude, (4) heading, (5) speed, and (6) time.

In one instance, the system 100 can also collect the real-time probe data and/or sensor data from one or more user equipment (UE) 109a-109n (also collectively referenced to herein as UEs 109) associated with the a vehicle 101 (e.g., an embedded navigation system), a user or a passenger of a vehicle 101 (e.g., a mobile device, a smartphone, etc.), or a combination thereof. In one instance, the UEs 109 may include one or more applications 111a-111n (also collectively referred to herein as applications 111) (e.g., a navigation or mapping application). In one instance, the system 100 may also collect the probe data and/or sensor data from one or more other sources such as government/municipality agencies, local or community agencies (e.g., a police department), and/or third-party official/semi-official sources (e.g., a services platform 117, one or more services 119a-119n, one or more content providers 121a-121m, etc.). In one embodiment, the probe data and/or sensor data collected by the vehicle sensors 103, the UEs 109, one or more other sources, or a combination thereof may be stored in the health/maintenance database 113, the geographic database 115, or a combination thereof.

Autonomous driving safety can require a vehicle to avoid high health risk vehicles in order to avoid a crash or fatality accident, to compensate for the deficiencies of the onboard senor data of one vehicle to react to the driving environment changes of neighboring vehicles and/or downstream vehicles. It will be much more effective to share information of high health risk vehicles by leveraging traffic channels and digital maps of the interconnected transport infrastructure that supports autonomous driving.

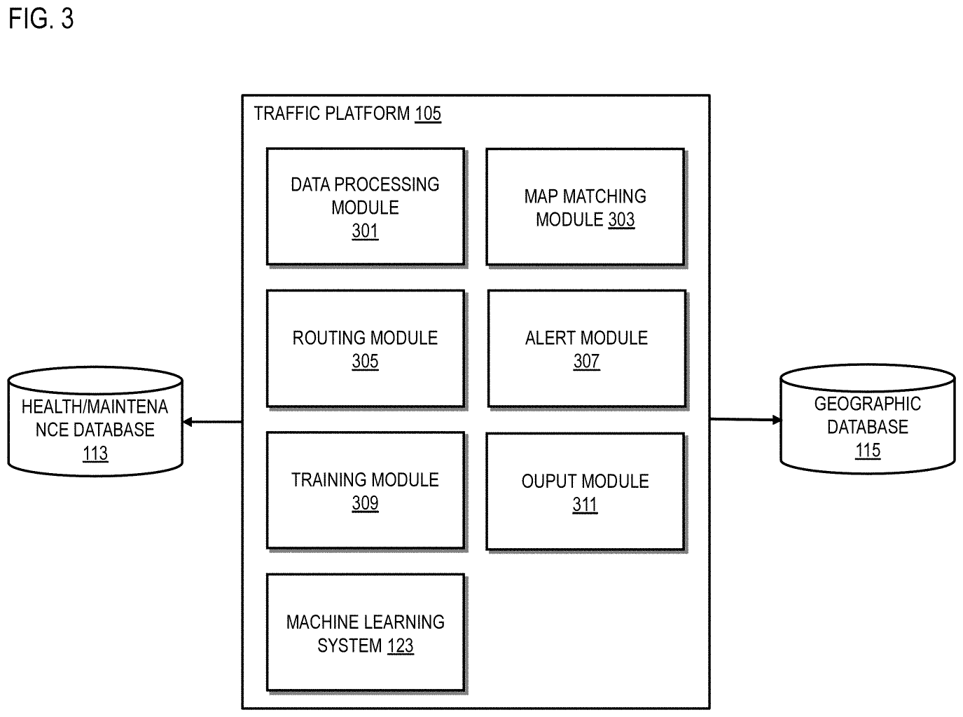

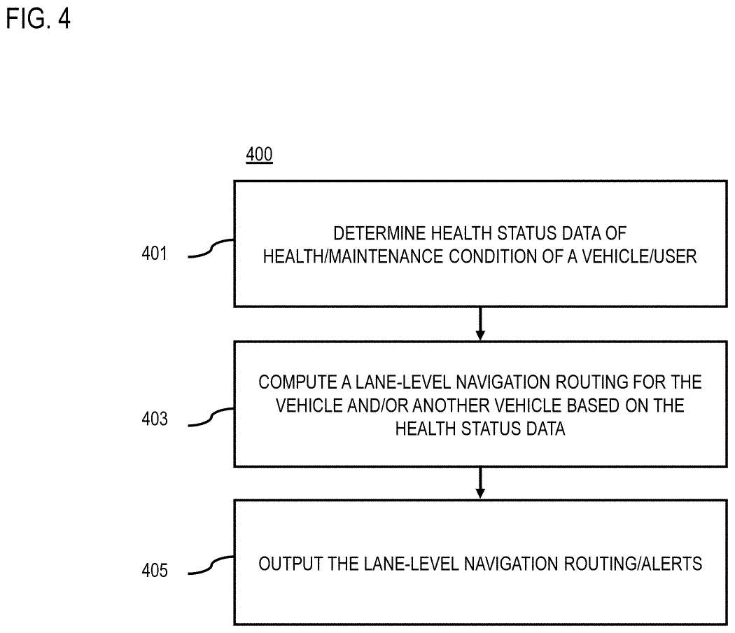

FIG. 3 is a diagram of the components of the traffic platform 105, according to one embodiment. By way of example, the traffic platform 105 includes one or more components for providing lane-level mapping/routing based on vehicle/user health data, according to the various embodiments described herein. It is contemplated that the functions of these components may be combined or performed by other components of equivalent functionality. In one embodiment, the traffic platform 105 includes a data processing module 301, a map-matching module 303, a routing module 305, an alert module 307, a training module 309, an output module 311, and a machine learning system 123 has connectivity to the health/maintenance database 113 and the geographic database 115. The above presented modules and components of the traffic platform 105 can be implemented in hardware, firmware, software, or a combination thereof. Though depicted as a separate entity in FIG. 1, it is contemplated that the traffic platform 105 may be implemented as a module of any other component of the system 100. In another embodiment, the traffic platform 105, the machine learning system 123, and/or the modules 301-311 may be implemented as a cloud-based service, local service, native application, or combination thereof. The functions of the traffic platform 105, the machine learning system 123, and/or the modules 301-311 are discussed with respect to FIG. 4.

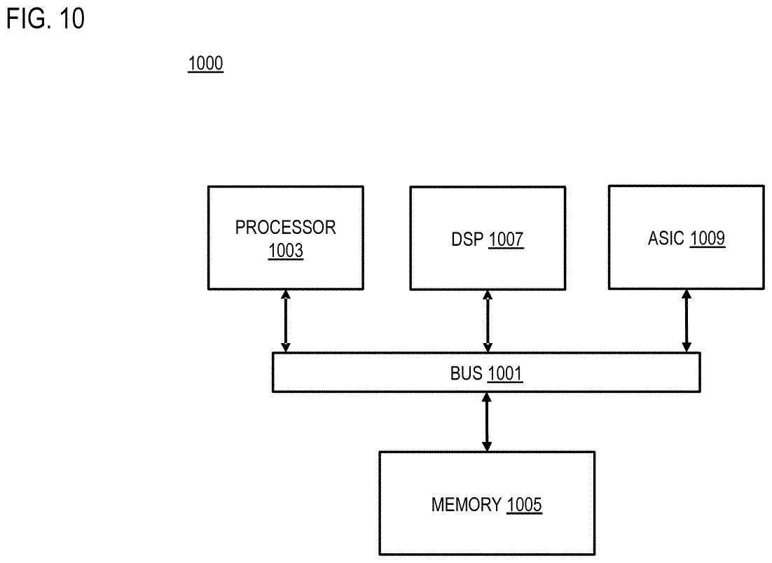

FIG. 4 is a flowchart of a process for providing lane-level mapping/routing based on vehicle/user health status data, according to one embodiment. In various embodiments, the traffic platform 105, the machine learning system 123, and/or any of the modules 301-311 may perform one or more portions of the process 400 and may be implemented in, for instance, a chip set including a processor and a memory as shown in FIG. 10. As such, the traffic platform 105 and/or the modules 301-311 can provide means for accomplishing various parts of the process 400, as well as means for accomplishing embodiments of other processes described herein in conjunction with other components of the system 100. Although the process 400 is illustrated and described as a sequence of steps, its contemplated that various embodiments of the process 400 may be performed in any order or combination and need not include all the illustrated steps. The process 400 can be carried by the traffic platform 105, the vehicle 101, or a combination thereof. In one embodiment, the traffic platform 105 executes the process 400 and sends a lane-level navigation routing and/or alert(s) to the vehicle 101 and/or other vehicle(s) (e.g., via the communication platform 107). In another embodiment, the vehicle 101 executes the process 400 and generates a lane-level navigation routing and/or alert(s) for itself and/or other vehicle(s) (e.g., via V2V communication services).

In one embodiment, in step 401, the data processing module 301 can determine health status data indicating at least one health or maintenance condition of a vehicle, a user of the vehicle, or a combination thereof. For instance, the health status data can be rated as Excellent, Good, Fair, and Poor. As another instance, the health status data can be rated as numbers 1 to 100.

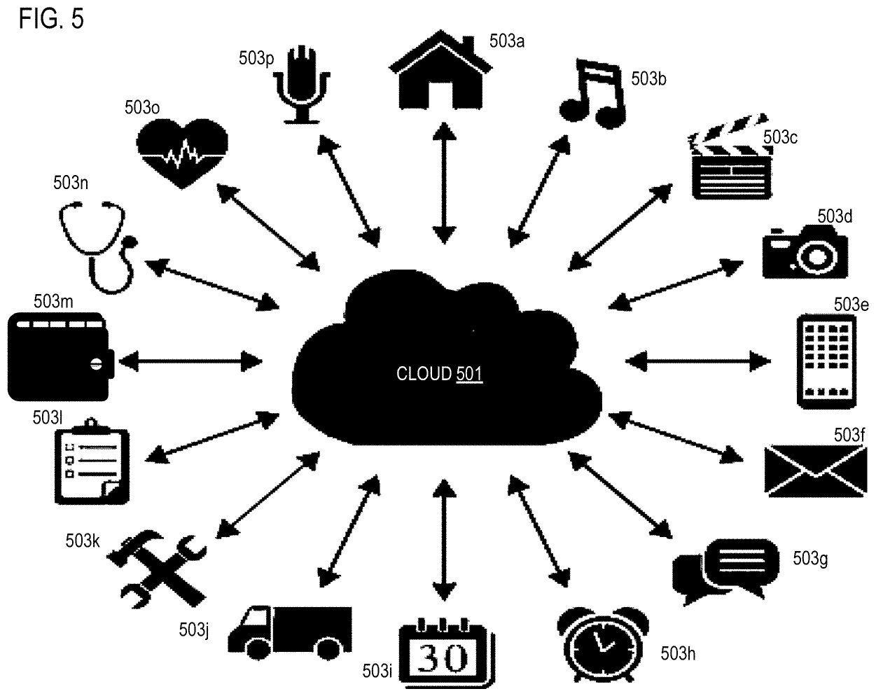

FIG. 5 is a diagram of a cloud-base vehicle/user heath or maintenance data infrastructure (e.g., a cloud 501), according to one embodiment. By way of example, the cloud 501 is connected with digital channels such as computing devices, mechanical and digital machines that are provided with unique identifiers (UIDs) and transfer vehicle/user heath or maintenance data over a network without requiring user interaction. The system 100 can invite vehicle owners and/or users to explicit opt in the heath or maintenance data collection via an user interface like selecting an opt in button or answering survey questions (e.g., for incentives, e.g., eligible to use a superhighway for sharing health data, insurance fee discounts, etc.). In another embodiment, the system 100 can collect the health data anonymously. By way of example, the system 100 can assign an anonymous ID number to each original vehicle/user ID number. This reassigned anonymous ID does not correspond to an individually identifiable account, yet can be tracked for heath or maintenance data. Once the anonymous file is created, the original vehicle/user ID numbers can be discarded. Alternatively, the original vehicle/user ID numbers can be truncated to the fewer digits to remain anonymous.

In one embodiment, the digital channels and/or sources of heath or maintenance data can include smart homes 503a, audio devices 503b, video devices 503c, cameras 503d, smart phones 503e, emails 503f, messages 503g (e.g., instant messages, social media chats/posts, blogs, etc.), digital clocks 503h, calendars 503i, modes of transport 503j (e.g., vehicle 101, bicycles, model, etc.), repair and maintenance services 503k (e.g., car body shop visits, engine oil changes, car insurance claims, third-party vehicle history reports, dealership recall service records, etc.), digital notes 5031, electronic wallets 503m, medical records 503n (e.g., hospital visits, tests, medications, etc.), fitness data 503o, smart speakers 503p, etc. By way of example, the smart homes 503a can include devices and appliances, e.g., lighting fixtures, thermostats, home security systems and cameras, and other home appliances. As another example, the fitness data 503o (e.g., sleep patterns, heart rates, blood pressure, exercises, etc.) can be collected form all kinds of fitness tracking devices, such as wearable devices communicating with the vehicle 101 and/or the system 100. In terms of sleep patterns, a sleep cycle is an oscillation between the slow-wave and REM (paradoxical) phases of sleep. The system 100 can analyze the sleep data to determine a quality of sleep and predict a driver's health status.

In one embodiment, the data processing module 301 can process vehicle location history data of the vehicle 101 (as a form of heath or maintenance data) to determine a vehicle service date, a distance driven by the vehicle since a service visit, or a combination thereof. By way of example, the health status data is determined based on the vehicle service date (e.g., Aug. 25, 2020), the distance driven (e.g., total mileage 71,113), or a combination thereof. In one embodiment, the vehicle location history data can be taken from the vehicle service centers (e.g., dealerships), if they use the branded centers. In one embodiment, the vehicle location history data can be taken from the user location information from Maps. For example, if there is an historical data in maps showing that the user's vehicle was in a local garage/service center for couple of hours/days. Then the system 100 can take the information from that database. All travel logs can be stored as GPS coordinates and the system 100 can take that information for each users/vehicle and use map data information to map-match which coordinates falls under garage/service centers. Using the last service details, the system 100 can predict the vehicle health status and store it in the health/maintenance database 113.

For instance, the processing of the health or maintenance data (e.g., vehicle location history data) involves the training module 309 and/or the machine learning system 123, and comprises using a trained predictive machine learning model to predict the health status data using the vehicle service date, the distance driven, the vehicle location history data, or a combination thereof as an input. By way of example, although sensor data shows the vehicle is healthy; however, the a trained predictive machine learning model uses historical data (e.g., missed oil changes) to predict that the vehicle engine will get wear and tear as to breakdown in the next half an hour. In this case, the routing module 305 and/or the alert module 307 will react to the prediction and generate lane-level navigation routing and respective alert for the driver. For example, the alert module 307 generates an alert: “Based on your health or the health of your car, please go to the right lane where other people are driving in approximately the same speed or have approximately the same health.”

In one embodiment, the location history data comprises location data collected from one or more sensors of the vehicle. Such location sensors can apply various positioning assisted navigation technologies, e.g., global navigation satellite systems (GNSS), WiFi, Bluetooth, Bluetooth low energy, 2/3/4/5/6G cellular signals, ultra-wideband (UWB) signals, etc., and various combinations of the technologies to derive a more precise location. By way of example, a combination of satellite and network signals can derive a more precise location than either one of the technologies, which is important in many of the intermodal scenarios, e.g., when GNSS signals are unavailable in tunnels.

In another embodiment, the data processing module 301 can process vehicle insurance data (as a form of heath or maintenance data) of the vehicle 101 to determine a vehicle service date, a distance driven by the vehicle since a service visit, or a combination thereof. By way of example, certain vehicle model/type is accident-prone, at certain age has great likelihood of failure/breakdown, etc.

In another embodiment, the data processing module 301 can process calendar data, user location history data, or a combination thereof associated with the user of the vehicle to determine a user sleep time, a user work time, a health check-up date of the user, a current health condition of the user, or a combination thereof. In this case, the health status data is based on the user sleep time, the user work time, the health check-up date, the current health condition, or a combination thereof. For instance, the health check-up date may be extracted from health insurance data, medical care data, fitness data, driver input data, police report data, crowdsourced data, video monitoring data, or a combination thereof of the user.

In one embodiment, the processing of the calendar data and/or the user location history data involves the training module 309 and/or the machine learning system 123, and comprises using a trained predictive machine learning model to predict the user sleep time, the user work time, the health check-up date, the current health condition, the calendar data, the user location history data, or a combination thereof as an input. By way of example, although sensor data shows the driver is healthy; however, the a trained predictive machine learning model uses historical data (e.g., scheduled prescription medication) to predict that the driver will get drowsy in the next half an hour. In this case, the routing module 305 and/or the alert module 307 will react to the prediction and generate lane-level navigation routing and respective alert for the driver.

Along with vehicle health status, the system 100 can store users health information and also keeping track on users last activities to predict how healthy and active the user is. For example, if the user had been in a disco pub all night and is travelling in early morning to some location without enough sleep, the user health can be marked as low and will be stored on in the health/maintenance database 113, thus the user will be navigated to a dedicated lane and will be provided with additional care.

In one embodiment, the map-matching module 303 can map-match real-time probe data of a vehicle 101 retrieved from a database (e.g., a local database of the vehicle 101a) with map data retrieved from a database (e.g., the geographic database 115) to see which road segment the vehicle 101a is travelling on (e.g., the middle lane). In addition, the map-matching module 303 can map-match probe data to a lane of the road segment. In one embodiment, the probe data is directly collected from one or more sensors (e.g., location sensors) of the vehicle 101 and/or UE 109 that traversed the road segment of a multi-lane roadway within the road network. In another embodiment, the probe data is retrieved from one or more probe data providers (e.g., content providers 121a-121m). As mentioned, probe data may be reported as probe points, which are individual data records collected at a point in time that records telemetry data for that point in time.

By way of example, the probe trajectory may be map-matched to the respective map or geographic records (e.g., stored in the geographic database 115) via location data associations (such as using known or future map matching or geo-coding techniques) and more specifically, the probe trajectory may be map-matched to individual lanes (e.g., any of the travel lanes, shoulder lanes, restricted lanes, service lanes, etc.) of the roadways by matching the geographic coordinates (e.g., longitude and latitude) between the probe data and the respective lanes of the road segment.

In another embodiment, the map-matching module 303 can apply the same map matching process using location sensor data to a lane of a road segment, such that the map-matching module 303 can map-match the health status data to the lane-level. The sensor data is collected from one or more sensors of at least one vehicle 101 (and/or other sensor data (e.g., from a UE 109) when traversing the road segment. In contrast to the probe data formatted as probe points, the sensor data is formatted as outputs from sensors of the vehicle 101 and/or the UE 109. By way of example, a speed sensor outputs a vehicle speed directly, while a vehicle speed value of a probe trajectory can be calculated by measuring a travel time between two probe data points of the probe trajectory.

In one embodiment, the health or maintenance data in FIG. 3 (e.g., a smart speaker, a wearable, etc.) can be reported via a smart vehicle anonymously, then the system 100 can map-match the health or maintenance data like probe data to find where the vehicle 100 at issue is and report it without privacy issue. By way of example, the system 10 collected 10 driver drowsy events from different smart vehicles reported this way. In another embodiment, the map-matching module 303 can map the health or maintenance data to digital map data of the geographic database 115, and provide the mapped health status data anonymously as a layer over the digital map to the output module 311 as later explained with FIGS. 5-7.

In one embodiment, in step 403, the routing module 305 can compute a lane-level navigation routing for the vehicle (e.g., vehicle 101a in FIG. 2A), another vehicle (e.g., vehicle 101b in FIG. 2A), or a combination thereof based on the health status data. In one embodiment, the lane-level navigation routing is computed by a routing engine using a cost function to minimize a safety risk indicated by the health status data, to optimize traffic associated with the health status data, or a combination thereof.

In one embodiment, the routing engine can calculate the lane level routing for traffic optimization. For example, when determining certain vehicles likely having maintenance issues, the routing engine can route these vehicles all to the right lane (e.g., at lower speeds), such that these vehicles can stop on the shoulder in case of breakdown or exit the road easily in need of assistance. This right-lane routing improve traffic by clearing up the other lanes for other vehicles to move faster without worrying issues caused by poorly-maintained vehicles. For instance, a poorly-maintained vehicle may suddenly breakdown on the left/fast lane, or has to move all the way from the left lane to exit the road due to maintenance issues. As another example, the system 100 can create a dedicated/special lane for this type of vehicles or grouping these vehicles with different issues in certain lanes for traffic optimization. Meanwhile, the alert module 307 can generate one or more alters of high health risk vehicles (e.g., vehicle 101b in FIG. 2A) based on the health status data and/or the lane-level navigation routing.

In one embodiment, the lane-level navigation routing provides guidance on which lane of a multi-lane road segment that the vehicle (e.g., vehicle 101a in FIG. 2A), the another vehicle (e.g., vehicle 101b in FIG. 2A), or a combination thereof should drive based on the health status data. For instances, the lane-level navigation routing (e.g., by the vehicle and/or the other vehicle) can include shifting to a different lane (e.g., FIG. 2A), exiting the road (e.g., FIG. 2B), taking a service road (e.g., FIG. 2C), or a combination thereof to avoid traffic accidents.

In one embodiment, the lane-level navigation routing provides guidance on which lane to drive on for the other vehicle to avoid the vehicle based on the health status data. For instance, the lane-level navigation routing is computed using a cost function to balance between re-routing cost (e.g., time, fuel consumption, vehicle wear and tear, road wear and tear, availability of gas stations, etc.) and a safety risk indicated by the health status data (e.g., potential collisions caused by a high health risk vehicle/driver). Referring back to FIG. 2A, since there is only one high health risk vehicle 101b ahead, the data processing module 301 can re-route either the vehicle 101a or the high health risk vehicle 101b to shift one lane. On the other hand, since there are a few high health risk vehicles 101b, 101c, 101d ahead in FIG. 2B, it is more efficient for the vehicle 101a to exit the road.

In one embodiment, the routing module 305 can monitor the at least one traffic accident risk, and update the lane-level navigation routing based, at least in part, on the monitoring. By way of example, the health status data of the vehicle 101b of FIG. 2A is updated to from a safety risk score of 86 to a lower safety risk score of 33 after the vehicle 101b stopped over a highway rest area. The routing module 305 updates the lane-level navigation routing for another vehicle 101c behind the vehicle 101b as “no action required” and no alert is issued to the vehicle 101c.

In one embodiment, in step 405, the output module 311 can provide the lane-level navigation routing and/or the alerts of the high health risk vehicle(s) as an output. For instance, the output can be a lane-level navigation routing to move a vehicle in an autonomous mode, or to recommend moving a vehicle in a manually driving mode to a certain lane for traffic optimization. In another embodiment, the output module 311 can process the output to generate an alert of the high health risk, to switch the high health risk vehicle 101b in FIG. 2A into an autonomous mode, to alert drivers of the high health risk vehicle 101b and its neighboring vehicles, to alert passengers of these vehicles (e.g., private vehicles, shared vehicles, taxis, buses, etc.), or a combination thereof.

In one embodiment, the output module 311 can deliver the lane-level navigation routing and/or the alerts of the high health risk vehicle(s) over the air radio interface, transport protocol experts group (TPEG) service by connected hypertext transfer protocol (HTTP) or user datagram protocol (UDP), and/or dedicated short range communications (DSRC) broadcasting data (e.g., via the communication network 107). In one instance, the system 100 can deliver the lane-level navigation routing and/or the alerts of the high health risk vehicle(s) to a vehicle 101, a user of the vehicle 101 (e.g., a driver or a passenger), or a combination thereof via a UE 109 (e.g., an embedded navigation system, a mobile device, or a combination thereof) and/or an application 111 running on the UE 109. In one example, a governmental agency (e.g., a police force) can use the lane-level navigation routing and/or the alerts of the high health risk vehicle(s) to better position service patrol resources (i.e., highway helper trucks). In another instance, a service provider (e.g., a service 119) can alert all mobile phone users with the lane-level navigation routing and/or the alerts of the high health risk vehicle(s) in a targeted area (e.g., through geofencing) using an emergency messaging system (e.g., the communication network 107). Consequently, the provision of the lane-level navigation routing and/or the alerts of the high health risk vehicle(s) to users can improve driver and vehicle awareness of the current vehicle/user health status states in the road network and take actions accordingly. In addition, in one embodiment, the lane-level navigation routing and/or the alerts of the high health risk vehicle(s) can be further used to improve autonomous driving safety.

The outputs of the health status data, the lane-level navigation routing, the alerts of the high health risk vehicle(s), the health status map data layer, etc. can be stored in the health/maintenance database 113 and/or the geographic database 115, and provide to users as a free service, an incentive to opt in health or maintenance data, or a paid service.

In one embodiment, the training module 309 in connection with the machine learning system 123 selects respective health or maintenance data factors (e.g., 503a-503p in FIG. 5) such as smart homes 503a, audio devices 503b, video devices 503c, cameras 503d, smart phones 503e, emails 503f, messages 503g (e.g., instant messages, social media chats/posts, blogs, etc.), digital clocks 503h, calendars 503i, modes of transport 503j (e.g., vehicle 101, bicycles, etc.), repair and maintenance services 503k (e.g., car body shop visits, engine oil changes, car insurance claims, third-party vehicle history reports, dealership recall service records, etc.), digital notes 5031, electronic wallets 503m, medical records 503n, fitness records 503o, smart speakers 503p, etc., to determine a relevel health status for a particular vehicle/user. In one embodiment, the training module 309 can train the machine learning system 123 to select or assign respective weights, correlations, relationships, etc. among the factors, to predict the most probable health status for the particular vehicle/user on at a lane, road segment, road, city, state, or country level. In one instance, the training module 309 can continuously provide and/or update a machine learning model (e.g., a support vector machine (SVM), neural network, decision tree, etc.) of the machine learning system 123 during training using, for instance, supervised deep convolution networks or equivalents. In other words, the training module 309 trains the machine learning model using the respective weights of the factors to predict the most probable health status most effectively and/or efficiently for the particular vehicle/user on at a desired level.

In another embodiment, the machine learning system 123 of the traffic platform 105 includes a neural network or other machine learning system to update (e.g., iteratively) health status data on lanes, road segments, etc., as well as the respective lane-level navigation routing. In one embodiment, the neural network of the machine learning system 123 is a traditional convolutional neural network which consists of multiple layers of collections of one or more neurons (which are configured to process a portion of an input data). In one embodiment, the machine learning system 123 also has connectivity or access over the communication network 107 to the health/maintenance database 113 and/or the geographic database 115 that can each store probe data, labeled or marked features (e.g., historically expected volumes and/or real-time actual observed volumes on road segments), health status data, lane-level navigation routing data, alerts, etc.

In one embodiment, the training module 309 can improve the lane-level mapping/routing process using feedback loops based on, for example, user behavior and/or feedback data. In one embodiment, the training module 309 can improve a machine learning model for the lane-level mapping/routing process using user behavior and/or feedback data as training data. For example, the training module 309 can correctly analyze health status data, missed health status data, etc. to determine the performance of the machine learning model.

In another embodiment, the system 100 can aggregate health status data (e.g., via the cloud 501 in FIG. 5) from a plurality of vehicles, a plurality of users of the plurality of vehicles, or a combination thereof. By way of example, the health status data indicates at least one health or maintenance condition of the plurality of vehicle, the plurality of users, or a combination thereof. The system 100 can map-match the aggregated health status data based on location data associated with the plurality of vehicles, the plurality of users, or a combination thereof. In one embodiment, the system 100 can generate a health status map data layer based on the map-matched health status data, and provide the health status map data layer as an output. Such health status map data layer can based generated based on real-time health status data, historical health status data, or a combination thereof.

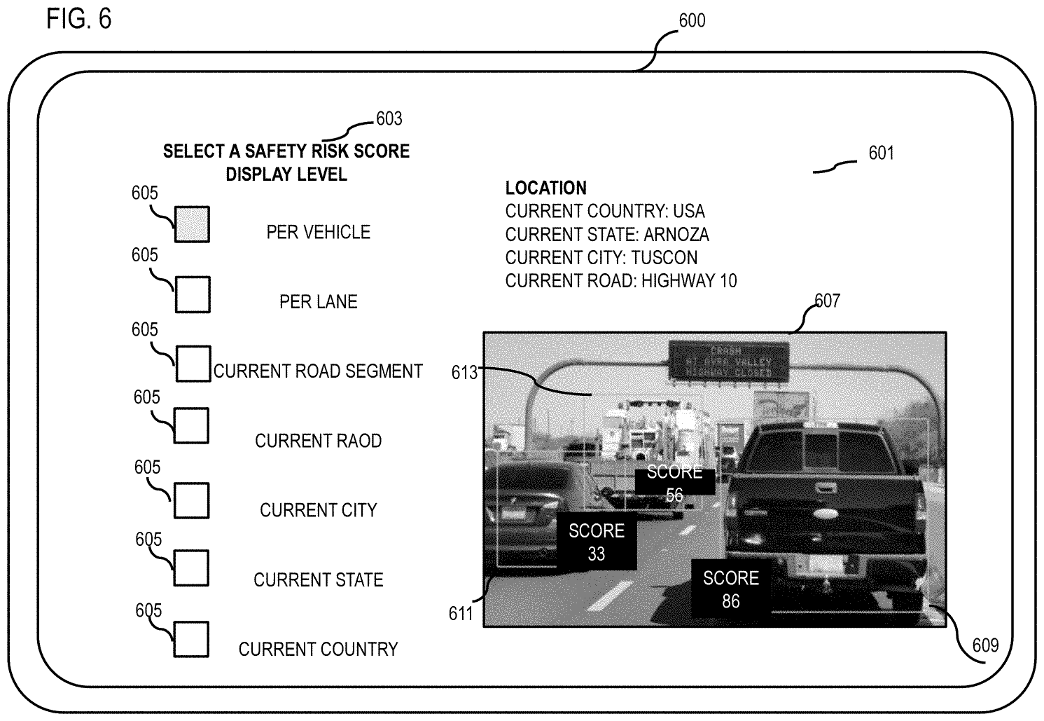

In one embodiment, the lane-level navigation routing is generated to reduce a safety risk, an accident risk, or a combination thereof. FIG. 6 is a diagram of an example user interface 600 for setting a safety risk score display level, according to various embodiments. FIG. 6 depicts a current location 601: Country: USA, State: Arizona, City: Tucson, and Road: Highway 10. FIG. 6 also depicts a user instruction 603: “Select a Safety Risk Score Display Level,” and the options 605 of per vehicle, per lane, current road segment, current road, current city, current state, current country, etc. After the selection of “per vehicle”, FIG. 6 shows a live image 607 taken by a vehicle camera (e.g., of the vehicle 101a of FIG. 2A).

The live image 607 shows each vehicle in front of the vehicle 101a in a bounding box with a safety risk score tag. For instances, a truck tracked in a bounding box 609 has a safety risk score of “86,” a passenger vehicle tracked in a bounding box 611 has a safety risk score of “33,” and a utility truck tracked in a bounding box 613 has a safety risk score of “56.” In this embodiment, the lower the safety risk score, the safer the vehicle/driver is. By way of example, the truck (scored “86”) on the middle lane has not had their oil change in the last 12 months, and the driver of the truck had less than four hours of sleep last night.

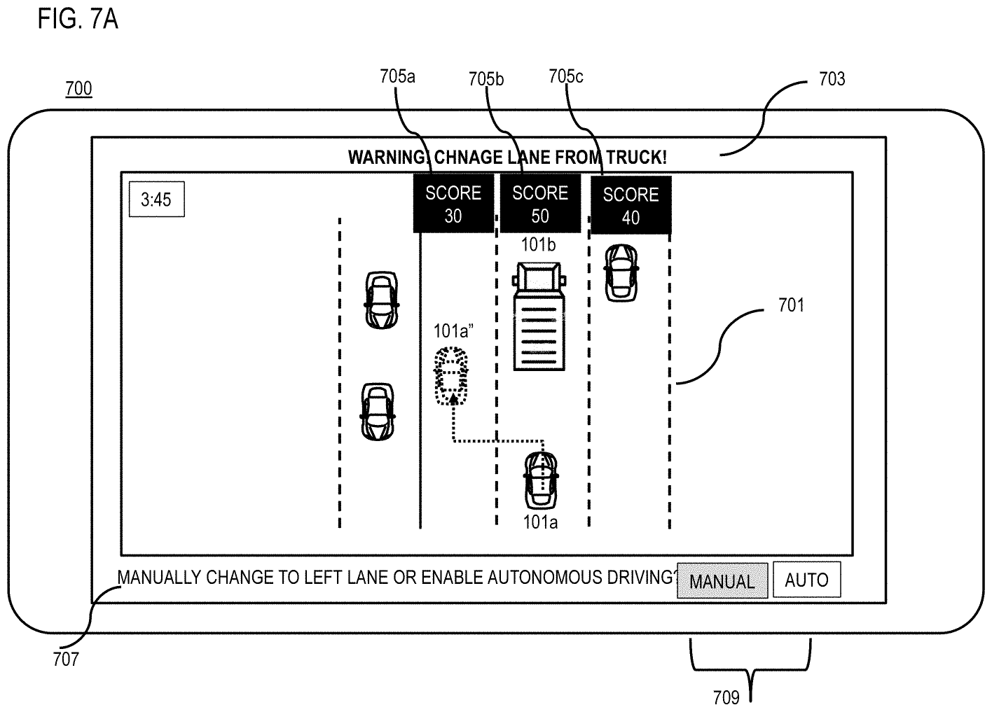

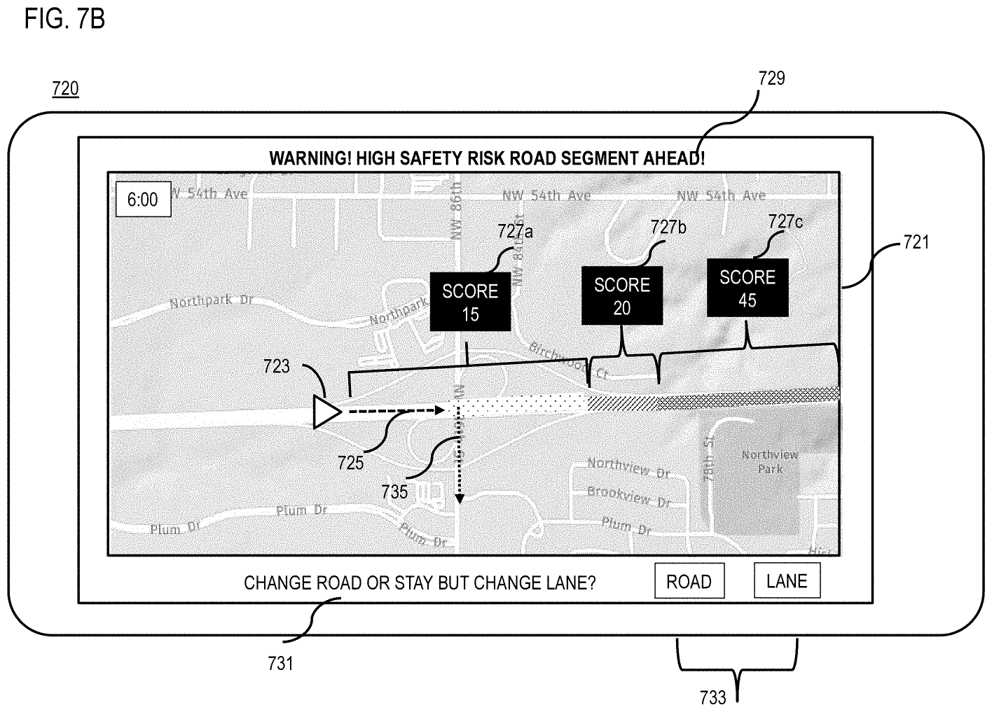

In one embodiment, the health status map data layer is used to provide lane-level navigation routing. After the safety risk score display level setting mode in FIG. 6, the user interface can be switched into a routing mode in FIGS. 7A-7B. FIGS. 7A-7B are diagrams of example user interfaces for presenting safety risk scores at different display levels in a routing mode, according to various embodiments.

In one embodiment, the system 100 can compute a lane-level safety risk score for one or more lanes of a road segment based on the map-matched health status data. By way of example, a lane-level safety risk score can be aggregated from safety risk scores of vehicles traveling on a lane segment, while a lane segment can be defined as one or more vehicle lengths (e.g., two-vehicle length of lane segment). FIG. 7A shows a user interface 700 for presenting safety risk scores at a lane level in the routing mode, according to one embodiment. Referring back to FIG. 2A, the system 100 determines the presence of a high health risk vehicle 101b in front of the vehicle 101a, and the options of changing to the right or left lanes from the middle lane of the path 201. FIG. 7A additionally looks into lane-level safety risk, by showing a diagram 701 of the lane-level mapping/routing event and an alert or instruction 703: “Warning! Change lane from truck!”.

In one embodiment, the health status map data layer further includes lane-level safety risk scores. FIG. 7A also shows a safety risk score tag 705a of “30” of the left lane, a safety risk score tag 705b of “50” of the middle lane, and a safety risk score tag 705c of “40” of the right lane. By way of example, 50% of the drivers on the middle lane have less than four hours of sleep last night, or 50% of the vehicles on the middle lane have not had their oil change in the last 6 months.

In this case, the system 100 can route the vehicle 101a to change to the left lane with a lower safety risk score, to avoid the most risky lane the vehicle 101a is currently on. FIG. 7A further shows an instruction 707: “Manually change to left lane of enable autonomous driving?” and buttons 709 of “Manual” and “Auto” for the user to select.

In one embodiment, the system 100 can compute a road-segment-level safety risk score for one or more road segments based on the map-matched health status data. FIG. 7B shows a user interface 720 for presenting safety risk scores at a road segment level in the routing mode, according to one embodiment. By way of example, a road segment safety risk score can be aggregated from lane-level safety risk scores of lanes in the road segment, while a road segment can be defined as one or more vehicle lengths (e.g., two-vehicle length of road segment). FIG. 7B looks into road-segment-level safety risk, by showing a map 721 and a vehicle 723 driving on a road 725 with three segment tagged with respective safety risk scores: a safety risk score tag 727a of “15” of the first road segment, a safety risk score tag 727b of “20” of the middle road segment, and a safety risk score tag 727c of “45” of the last road segment. By way of example, 45% of the drivers on the last road segment (e.g., a few wine bars) were drowsy between 1:00-2:30 am on weekend, to avoid the road segment with high chance of accidents during the time frame, the system 100 can offer a lane-level routing or to change to a different road. The same approach can be applied to streets near hospitals, e.g., people in a hospital working overtime, so route around the hospital during certain time periods just to be safe.

In one embodiment, the health status map data layer further includes road-segment-level safety risk scores. FIG. 7B also shows an alert or instruction 729: “Warning! High health risk road segment ahead!”, another alert or instruction 731: “Change road or Stay but change lane?”, buttons 733 of “Road” and “Lane” for the user to select. When the “Road” button is selected, the system 100 can route the vehicle 723 to a different road 735 with a lower safety risk score, to avoid the high health risk road segment on the road 725. When the “Lane” button is selected, the system 100 can route the vehicle 723 to a different lane on the road 725 with a lowest safety risk score regardless the vehicle 723 is traveling on which of the road segments on the road 725.

By analogy, the system 100 can compute a road-level safety risk score, a city-level safety risk score, a state-level safety risk score, a country-level safety risk score, etc. based on the map-matched health status data directly, or by aggregating lower level safety risk scores. In other embodiments, the health status map data layer further includes the road-level safety risk score, the city-level safety risk score, the state-level safety risk score, the country-level safety risk score, etc.

In one embodiment, the system 100 can publish the output (e.g., the health status data, the lane-level navigation routing, the alerts/instructions, the safety risk scores, the health status map data layer, etc.) in a health or maintenance database (e.g., the health/maintenance database 113), a geographic database (e.g., the geographic database 115), a location-based service, a health/maintenance data cloud (e.g., cloud 501), or a combination thereof. In one embodiment, the system 100 can cause the vehicles to submit health or maintenance data to the a health or maintenance database, the geographic database, the location-based service, the health/maintenance data cloud, or a combination thereof. The anonymous health or maintenance data can be tagged with location data.

In one embodiment, the health status data can be predicted from historical location data associated with the plurality of vehicles, the plurality of users, or combination thereof using a machine learning model, as discussed in conjunction with FIG. 5.

In one embodiment, the system 100 can perform a predictive analysis based on the map-matched health status data to determine which of the at least one health or maintenance condition is predicted to occur in a lane or a road segment. In another embodiment, In one embodiment, the system 100 can perform a predictive analysis based on the map-matched health status data to predict traffic accidents and/or traffic flows.

The system 100 can map the health status data, and/or the safety risk scores into the health/maintenance database 113 and/or the geographic database 115 as a data layer as well as overlay onto a digital map to show the risky lanes/road segments/roads/etc. In addition, the system 100 can use the data layer for accident prediction and traffic modeling.

In addition to avoid/mitigate traffic accidents, the system 100 can optimize the lane use to maintain traffic flow and accommodate slow/bad vehicles (e.g., with vehicle maintenance/health issue). Taking FIG. 2A as an example, the system 100 not only reduces the accident risk for the slow/bad vehicle in the middle lane, but also moves the traffic on the right/left lanes quicker.

In yet another embodiment, the system 100 can receive health status data associated with a vehicle, a user of the vehicle, or a combination thereof traveling in a road network. The system 100 can process the health status data to predict a safety risk, an accident risk, or a combination thereof associated with the vehicle, the user, or a combination thereof at a lane level of the road network. The system 100 can provide data for broadcasting the predicted safety risk, the predicted accident risk, or a combination thereof via a communication channel (e.g., traffic channels, digital channels, etc.).

The terms safety and risk refer to two separate but related aspects of traffic accidents. Safety refers to a current condition within a vehicle or a user, or on the road or the road network. Safety considers whether or not there is an immediate threat of danger to the vehicle or the user. A threat of danger refers to a specific traffic situation that is out of control, imminent, and likely to have severe effects on the vehicle or the user. A user is assessed to be safe when there is no threat of danger within the vehicle, or on the road or the road network. When such a threat exists, the vehicle can have sufficient protective capacities to protect the user and manage the threat.

Risk refers to the likelihood of accidents occurring in the future. An assessment of risk includes the identification of risk factors, which are vehicle/driver behaviors and conditions that create an environment or circumstances that increase the chance of accident. Risk factors of various degrees and seriousness may exist within the vehicle or on the road/road network.

By way of example, traffic channels may include multiple traffic message channels (TMCs), vehicle-to-vehicle (V2V) communication services, vehicle-to-everything (V2X) communication services, etc. The digital channels may include the computing devices, the mechanical and digital machines that are provided with unique identifiers (UIDs) and transfer vehicle/user heath or maintenance data over a network without requiring user interaction as shown in FIG. 5.

In one embodiment, the predicted safety risk, the predicted accident risk, or a combination thereof is used to compute a lane-level navigation routing within the road network. In another embodiment, the lane-level navigation routing comprises shifting to a different lane, taking an exit, taking an alternate road segment, or a combination thereof (e.g., by the vehicle 101a and/or vehicle 101b in FIG. 2A), to reduce the predicted safety risk, the predicted accident risk, or a combination thereof. By way of example, the health status data can be predicted from historical location data associated with the vehicle, the user, or combination thereof using a machine learning model.

The above-discussed embodiments can provide one or more digital channels to predict or provide vehicle/user health status data to route the vehicle at a lane-level and avoid traffic accidents. Optionally, the vehicle/user health status data is map-matched into a digital map for broadcasting via the digital channels and/or traffic channels. The digital channels collect/use vehicle health status data (e.g., determined based on when was the last servicing done, based on of total kms/miles driven/last garage visit based on Location history etc. can be used) and user health status data (e.g., determined based on sleeping time/working time/last health check-up/current health conditions etc.).

The above-discussed embodiments can alert one or more upstream vehicles to take one or more strategies to avoid high health risk vehicles, such as changing to a different lane, exiting the road, using a service road, etc. The above-discussed embodiments can alert high health risk drivers (e.g., sick, or tired after 15-hr shift) to let autonomous vehicles to take over, or alert drivers of high health risk vehicles (e.g., likely to breakdown) to swap vehicles if possible.

Returning to FIG. 1, in one embodiment, the traffic platform 105 has connectivity over the communication network 107 to the services platform 117 (e.g., an OEM platform) that provides one or more services 119a-119n (also collectively referred to herein as services 119) (e.g., probe and/or sensor data collection services). By way of example, the services 119 may also be other third-party services and include mapping services, navigation services, traffic incident services, travel planning services, notification services, social networking services, content (e.g., audio, video, images, etc.) provisioning services, application services, storage services, contextual information determination services, location-based services, information-based services (e.g., weather, news, etc.), etc. In one embodiment, the services platform 117 uses the output (e.g. lane-level lane departure event detection and messages) of the traffic platform 105 to provide services such as navigation, mapping, other location-based services, etc.

In one embodiment, the traffic platform 105 may be a platform with multiple interconnected components. The traffic platform 105 may include multiple servers, intelligent networking devices, computing devices, components, and corresponding software for providing parametric representations of lane lines. In addition, it is noted that the traffic platform 105 may be a separate entity of the system 100, a part of the services platform 117, a part of the one or more services 119, or included within the vehicles 101 (e.g., an embedded navigation system).

In one embodiment, content providers 121a-121m (also collectively referred to herein as content providers 121) may provide content or data (e.g., including probe data, sensor data, etc.) to the traffic platform 105, the UEs 109, the applications 111, the health/maintenance database 113, the geographic database 115, the services platform 117, the services 119, and the vehicles 101. The content provided may be any type of content, such as map content, textual content, audio content, video content, image content, etc. In one embodiment, the content providers 121 may provide content that may aid in localizing a vehicle path or trajectory on a lane of a digital map or link. In one embodiment, the content providers 121 may also store content associated with the traffic platform 105, the health/maintenance database 113, the geographic database 115, the services platform 117, the services 119, and/or the vehicles 101. In another embodiment, the content providers 121 may manage access to a central repository of data, and offer a consistent, standard interface to data, such as a repository of the geographic database 115.

By way of example, the UEs 109 are any type of embedded system, mobile terminal, fixed terminal, or portable terminal including a built-in navigation system, a personal navigation device, mobile handset, station, unit, device, multimedia computer, multimedia tablet, Internet node, communicator, desktop computer, laptop computer, notebook computer, netbook computer, tablet computer, personal communication system (PCS) device, personal digital assistants (PDAs), audio/video player, digital camera/camcorder, positioning device, fitness device, television receiver, radio broadcast receiver, electronic book device, game device, or any combination thereof, including the accessories and peripherals of these devices, or any combination thereof. It is also contemplated that a UE 109 can support any type of interface to the user (such as “wearable” circuitry, etc.). In one embodiment, a UE 109 may be associated with a vehicle 101 (e.g., a mobile device) or be a component part of the vehicle 101 (e.g., an embedded navigation system). In one embodiment, the UEs 109 may include the traffic platform 105 to provide lane-level mapping/routing based on vehicle/user health status data.

In one embodiment, as mentioned above, the vehicles 101, for instance, are part of a probe-based system for collecting probe data and/or sensor data for lane-level mapping/routing based on vehicle/user health data. In one embodiment, each vehicle 101 is configured to report probe data as probe points, which are individual data records collected at a point in time that records telemetry data for that point in time. In one embodiment, the probe ID can be permanent or valid for a certain period of time. In one embodiment, the probe ID is cycled, particularly for consumer-sourced data, to protect the privacy of the source.

In one embodiment, a probe point can include attributes such as: (1) probe ID, (2) longitude, (3) latitude, (4) heading, (5) speed, and (6) time. The list of attributes is provided by way of illustration and not limitation. Accordingly, it is contemplated that any combination of these attributes or other attributes may be recorded as a probe point. For example, attributes such as altitude (e.g., for flight capable vehicles or for tracking non-flight vehicles in the altitude domain), tilt, steering angle, wiper activation, etc. can be included and reported for a probe point. In one embodiment, the vehicles 101 may include sensors 103 for reporting measuring and/or reporting attributes. The attributes can also be any attribute normally collected by an on-board diagnostic (OBD) system of the vehicle 101, and available through an interface to the OBD system (e.g., OBD II interface or other similar interface).

The probe points can be reported from the vehicles 101 in real-time, in batches, continuously, or at any other frequency requested by the system 100 over, for instance, the communication network 107 for processing by the traffic platform 105. The probe points also can be map matched to specific road links stored in the geographic database 115. In one embodiment, the system 100 (e.g., via the traffic platform 105) can generate probe traces (e.g., vehicle paths or trajectories) from the probe points for an individual probe so that the probe traces represent a travel trajectory or vehicle path of the probe through the road network.

In one embodiment, as previously stated, the vehicles 101 are configured with various sensors (e.g., vehicle sensors 103) for generating or collecting probe data, sensor data, related geographic/map data, etc. In one embodiment, the sensed data represents sensor data associated with a geographic location or coordinates at which the sensor data was collected. In one embodiment, the probe data (e.g., stored in the geographic database 115) includes location probes collected by one or more vehicle sensors 103. By way of example, the vehicle sensors 103 may include a RADAR system, a LiDAR system, global positioning sensor for gathering location data (e.g., GPS), a network detection sensor for detecting wireless signals or receivers for different short-range communications (e.g., Bluetooth, Wi-Fi, Li-Fi, near field communication (NFC) etc.), temporal information sensors, a camera/imaging sensor for gathering image data, an audio recorder for gathering audio data, velocity sensors mounted on a steering wheel of the vehicles 101, switch sensors for determining whether one or more vehicle switches are engaged, and the like. Though depicted as automobiles, it is contemplated the vehicles 101 can be any type of vehicle manned or unmanned (e.g., cars, trucks, buses, vans, motorcycles, scooters, drones, etc.) that travel through road segments of a road network.

Other examples of sensors 103 of the vehicle 101 may include light sensors, orientation sensors augmented with height sensors and acceleration sensor (e.g., an accelerometer can measure acceleration and can be used to determine orientation of the vehicle), tilt sensors to detect the degree of incline or decline of the vehicle 101 along a path of travel (e.g., while on a hill or a cliff), moisture sensors, pressure sensors, etc. In a further example embodiment, sensors 103 about the perimeter of the vehicle 101 may detect the relative distance of the vehicle 101 from a physical divider, a lane line of a link or roadway (e.g., vehicle path 201), the presence of other vehicles, pedestrians, traffic lights, potholes and any other objects, or a combination thereof. In one scenario, the vehicle sensors 103 may detect weather data, traffic information, or a combination thereof. In one embodiment, the vehicles 101 may include GPS or other satellite-based receivers to obtain geographic coordinates from satellites 125 for determining current location and time. Further, the location can be determined by visual odometry, triangulation systems such as A-GPS, Cell of Origin, or other location extrapolation technologies.

In one embodiment, the UEs 109 may also be configured with various sensors (not shown for illustrative convenience) for acquiring and/or generating probe data and/or sensor data associated with a vehicle 101, a driver, other vehicles, conditions regarding the driving environment or roadway, etc. For example, such sensors may be used as GPS receivers for interacting with the one or more satellites 125 to determine and track the current speed, position, and location of a vehicle 101 travelling along a link or roadway. In addition, the sensors may gather tilt data (e.g., a degree of incline or decline of the vehicle during travel), motion data, light data, sound data, image data, weather data, temporal data and other data associated with the vehicles 101 and/or UEs 109. Still further, the sensors may detect local or transient network and/or wireless signals, such as those transmitted by nearby devices during navigation of a vehicle along a roadway (Li-Fi, near field communication (NFC)) etc.

It is noted therefore that the above described data may be transmitted via communication network 107 as probe data (e.g., GPS probe data) according to any known wireless communication protocols. For example, each UE 109, application 111, user, and/or vehicle 101 may be assigned a unique probe identifier (probe ID) for use in reporting or transmitting said probe data collected by the vehicles 101 and/or UEs 109. In one embodiment, each vehicle 101 and/or UE 109 is configured to report probe data as probe points, which are individual data records collected at a point in time that records telemetry data.

In one embodiment, the traffic platform 105 retrieves aggregated probe points gathered and/or generated by the vehicle sensors 103 and/or the UE 109 resulting from the travel of the UEs 109 and/or vehicles 101 on a road segment of a road network. In one instance, the geographic database 115 stores a plurality of probe points and/or trajectories generated by different vehicle sensors 103, UEs 109, applications 111, vehicles 101, etc. over a period while traveling in a monitored area. A time sequence of probe points specifies a trajectory—i.e., a path traversed by a UE 109, application 111, vehicle 101, etc. over the period.

In one embodiment, the communication network 107 of the system 100 includes one or more networks such as a data network, a wireless network, a telephony network, or any combination thereof. It is contemplated that the data network may be any local area network (LAN), metropolitan area network (MAN), wide area network (WAN), a public data network (e.g., the Internet), short range wireless network, or any other suitable packet-switched network, such as a commercially owned, proprietary packet-switched network, e.g., a proprietary cable or fiber-optic network, and the like, or any combination thereof. In addition, the wireless network may be, for example, a cellular network and may employ various technologies including enhanced data rates for global evolution (EDGE), general packet radio service (GPRS), global system for mobile communications (GSM), Internet protocol multimedia subsystem (IMS), universal mobile telecommunications system (UNITS), etc., as well as any other suitable wireless medium, e.g., worldwide interoperability for microwave access (WiMAX), Long Term Evolution (LTE) networks, code division multiple access (CDMA), wideband code division multiple access (WCDMA), wireless fidelity (Wi-Fi), wireless LAN (WLAN), Bluetooth®, Internet Protocol (IP) data casting, satellite, mobile ad-hoc network (MANET), and the like, or any combination thereof.

By way of example, the vehicles 101, vehicle sensors 103, traffic platform 105, UEs 109, applications 111, services platform 117, services 119, content providers 121, and/or satellites 125 communicate with each other and other components of the system 100 using well known, new or still developing protocols. In this context, a protocol includes a set of rules defining how the network nodes within the communication network 107 interact with each other based on information sent over the communication links. The protocols are effective at different layers of operation within each node, from generating and receiving physical signals of various types, to selecting a link for transferring those signals, to the format of information indicated by those signals, to identifying which software application executing on a computer system sends or receives the information. The conceptually different layers of protocols for exchanging information over a network are described in the Open Systems Interconnection (OSI) Reference Model.

Communications between the network nodes are typically effected by exchanging discrete packets of data. Each packet typically comprises (1) header information associated with a particular protocol, and (2) payload information that follows the header information and contains information that may be processed independently of that particular protocol. In some protocols, the packet includes (3) trailer information following the payload and indicating the end of the payload information. The header includes information such as the source of the packet, its destination, the length of the payload, and other properties used by the protocol. Often, the data in the payload for the particular protocol includes a header and payload for a different protocol associated with a different, higher layer of the OSI Reference Model. The header for a particular protocol typically indicates a type for the next protocol contained in its payload. The higher layer protocol is said to be encapsulated in the lower layer protocol. The headers included in a packet traversing multiple heterogeneous networks, such as the Internet, typically include a physical (layer 1) header, a data-link (layer 2) header, an internetwork (layer 3) header and a transport (layer 4) header, and various application (layer 5, layer 6 and layer 7) headers as defined by the OSI Reference Model.

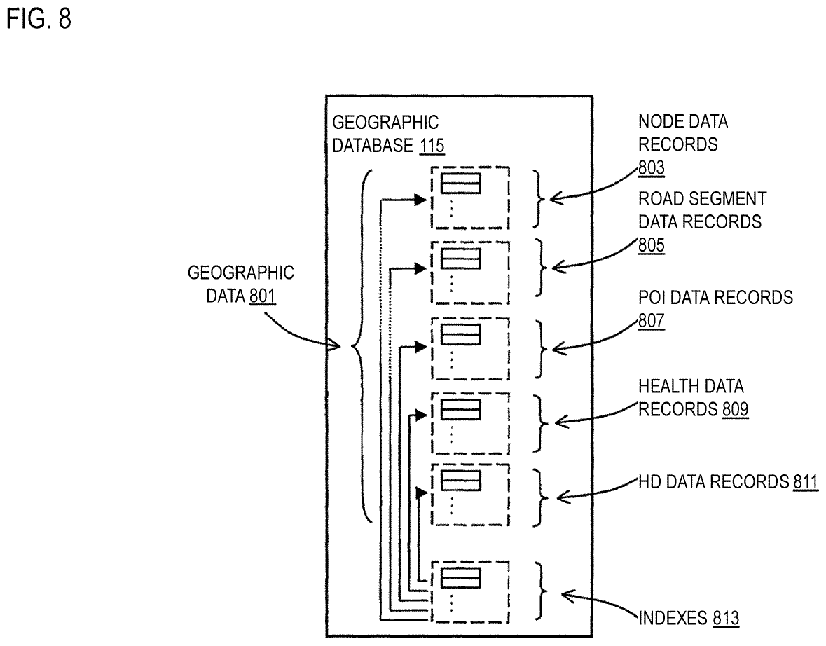

FIG. 8 is a diagram of a geographic database (such as the database 115), according to one embodiment. In one embodiment, the geographic database 115 includes geographic data 801 used for (or configured to be compiled to be used for) mapping and/or navigation-related services, such as for video odometry based on the parametric representation of lanes include, e.g., encoding and/or decoding parametric representations into lane lines. In one embodiment, the geographic database 115 include high resolution or high definition (HD) mapping data that provide centimeter-level or better accuracy of map features. For example, the geographic database 115 can be based on Light Detection and Ranging (LiDAR) or equivalent technology to collect billions of 3D points and model road surfaces and other map features down to the number lanes and their widths. In one embodiment, the HD mapping data (e.g., HD data records 811) capture and store details such as the slope and curvature of the road, lane markings, roadside objects such as signposts, including what the signage denotes. By way of example, the HD mapping data enable highly automated vehicles to precisely localize themselves on the road.

In one embodiment, geographic features (e.g., two-dimensional, or three-dimensional features) are represented using polygons (e.g., two-dimensional features) or polygon extrusions (e.g., three-dimensional features). For example, the edges of the polygons correspond to the boundaries or edges of the respective geographic feature. In the case of a building, a two-dimensional polygon can be used to represent a footprint of the building, and a three-dimensional polygon extrusion can be used to represent the three-dimensional surfaces of the building. It is contemplated that although various embodiments are discussed with respect to two-dimensional polygons, it is contemplated that the embodiments are also applicable to three-dimensional polygon extrusions. Accordingly, the terms polygons and polygon extrusions as used herein can be used interchangeably.

In one embodiment, the following terminology applies to the representation of geographic features in the geographic database 115.

“Node”—A point that terminates a link.

“Line segment”—A straight line connecting two points.

“Link” (or “edge”)—A contiguous, non-branching string of one or more line segments terminating in a node at each end.

“Shape point”—A point along a link between two nodes (e.g., used to alert a shape of the link without defining new nodes).

“Oriented link”—A link that has a starting node (referred to as the “reference node”) and an ending node (referred to as the “non reference node”).

“Simple polygon”—An interior area of an outer boundary formed by a string of oriented links that begins and ends in one node. In one embodiment, a simple polygon does not cross itself.

“Polygon”—An area bounded by an outer boundary and none or at least one interior boundary (e.g., a hole or island). In one embodiment, a polygon is constructed from one outer simple polygon and none or at least one inner simple polygon. A polygon is simple if it just consists of one simple polygon, or complex if it has at least one inner simple polygon.

In one embodiment, the geographic database 115 follows certain conventions. For example, links do not cross themselves and do not cross each other except at a node. Also, there are no duplicated shape points, nodes, or links. Two links that connect each other have a common node. In the geographic database 115, overlapping geographic features are represented by overlapping polygons. When polygons overlap, the boundary of one polygon crosses the boundary of the other polygon. In the geographic database 115, the location at which the boundary of one polygon intersects they boundary of another polygon is represented by a node. In one embodiment, a node may be used to represent other locations along the boundary of a polygon than a location at which the boundary of the polygon intersects the boundary of another polygon. In one embodiment, a shape point is not used to represent a point at which the boundary of a polygon intersects the boundary of another polygon.

As shown, the geographic database 115 includes node data records 803, road segment or link data records 805, POI data records 807, health data records 809, HD mapping data records 811, and indexes 813, for example. More, fewer, or different data records can be provided. In one embodiment, additional data records (not shown) can include cartographic (“carto”) data records, routing data, and maneuver data. In one embodiment, the indexes 813 may improve the speed of data retrieval operations in the geographic database 115. In one embodiment, the indexes 813 may be used to quickly locate data without having to search every row in the geographic database 115 every time it is accessed. For example, in one embodiment, the indexes 813 can be a spatial index of the polygon points associated with stored feature polygons.

In exemplary embodiments, the road segment data records 805 are links or segments representing roads, streets, or paths, as can be used in the calculated route or recorded route information for determination of one or more personalized routes. The node data records 803 are end points corresponding to the respective links or segments of the road segment data records 805. The road link data records 805 and the node data records 803 represent a road network, such as used by vehicles, cars, and/or other entities. Alternatively, the geographic database 115 can contain path segment and node data records or other data that represent pedestrian paths or areas in addition to or instead of the vehicle road record data, for example.

The road/link segments and nodes can be associated with attributes, such as geographic coordinates, street names, address ranges, speed limits, turn restrictions at intersections, and other navigation related attributes, as well as POIs, such as gasoline stations, hotels, restaurants, museums, stadiums, offices, automobile dealerships, auto repair shops, buildings, stores, parks, etc. The geographic database 115 can include data about the POIs and their respective locations in the POI data records 807. The geographic database 115 can also include data about places, such as cities, towns, or other communities, and other geographic features, such as bodies of water, mountain ranges, etc. Such place or feature data can be part of the POI data records 807 or can be associated with POIs or POI data records 807 (such as a data point used for displaying or representing a position of a city).

In one embodiment, the geographic database 115 can also include health data records 809 for storing the health status data, the lane-level navigation routing, the alerts of the high health risk vehicle(s), the health status map data layer, training data, prediction models, annotated observations, computed featured distributions, sampling probabilities, and/or any other data generated or used by the system 100 according to the various embodiments described herein. By way of example, the health data records 809 can be associated with one or more of the node records 803, road segment records 805, and/or POI data records 807 to support traffic reporting and/or autonomous driving based on the features stored therein and the corresponding estimated quality of the features. In this way, the health data records 809 can also be associated with or used to classify the characteristics or metadata of the corresponding records 803, 805, and/or 807.

In one embodiment, as discussed above, the HID mapping data records 811 model road surfaces and other map features to centimeter-level or better accuracy. The HD mapping data records 811 also include lane models that provide the precise lane geometry with lane boundaries, as well as rich attributes of the lane models. These rich attributes include, but are not limited to, lane traversal information, lane types, lane marking types, lane level speed limit information, and/or the like. In one embodiment, the HD mapping data records 811 are divided into spatial partitions of varying sizes to provide HD mapping data to vehicles 101 and other end user devices with near real-time speed without overloading the available resources of the vehicles 101 and/or devices (e.g., computational, memory, bandwidth, etc. resources).

In one embodiment, the HD mapping data records 811 are created from high-resolution 3D mesh or point-cloud data generated, for instance, from LiDAR-equipped vehicles. The 3D mesh or point-cloud data are processed to create 3D representations of a street or geographic environment at centimeter-level accuracy for storage in the HD mapping data records 811.

In one embodiment, the HD mapping data records 811 also include real-time sensor data collected from probe vehicles in the field. The real-time sensor data, for instance, integrates real-time traffic information, weather, and road conditions (e.g., potholes, road friction, road wear, etc.) with highly detailed 3D representations of street and geographic features to provide precise real-time also at centimeter-level accuracy. Other sensor data can include vehicle telemetry or operational data such as windshield wiper activation state, braking state, steering angle, accelerator position, and/or the like.

In one embodiment, the geographic database 115 can be maintained by the content provider 121 in association with the services platform 117 (e.g., a map developer). The map developer can collect geographic data to generate and enhance the geographic database 115. There can be different ways used by the map developer to collect data. These ways can include obtaining data from other sources, such as municipalities or respective geographic authorities. In addition, the map developer can employ field personnel to travel by vehicle (e.g., vehicles 101 and/or user terminals 109) along roads throughout the geographic region to observe features and/or record information about them, for example. Also, remote sensing, such as aerial or satellite photography, can be used.

The geographic database 115 can be a master geographic database stored in a format that facilitates updating, maintenance, and development. For example, the master geographic database or data in the master geographic database can be in an Oracle spatial format or other spatial format, such as for development or production purposes. The Oracle spatial format or development/production database can be compiled into a delivery format, such as a geographic data files (GDF) format. The data in the production and/or delivery formats can be compiled or further compiled to form geographic database products or databases, which can be used in end user navigation devices or systems.

For example, geographic data is compiled (such as into a platform specification format (PSF) format) to organize and/or configure the data for performing navigation-related functions and/or services, such as route calculation, route guidance, map display, speed calculation, distance and travel time functions, and other functions, by a navigation device, such as by a vehicle 101 or a user terminal 109, for example. The navigation-related functions can correspond to vehicle navigation, pedestrian navigation, or other types of navigation. The compilation to produce the end user databases can be performed by a party or entity separate from the map developer. For example, a customer of the map developer, such as a navigation device developer or other end user device developer, can perform compilation on a received geographic database in a delivery format to produce one or more compiled navigation databases.

The processes described herein for providing lane-level mapping/routing based on vehicle/user health status data may be advantageously implemented via software, hardware (e.g., general processor, Digital Signal Processing (DSP) chip, an Application Specific Integrated Circuit (ASIC), Field Programmable Gate Arrays (FPGAs), etc.), firmware or a combination thereof. Such exemplary hardware for performing the described functions is detailed below.

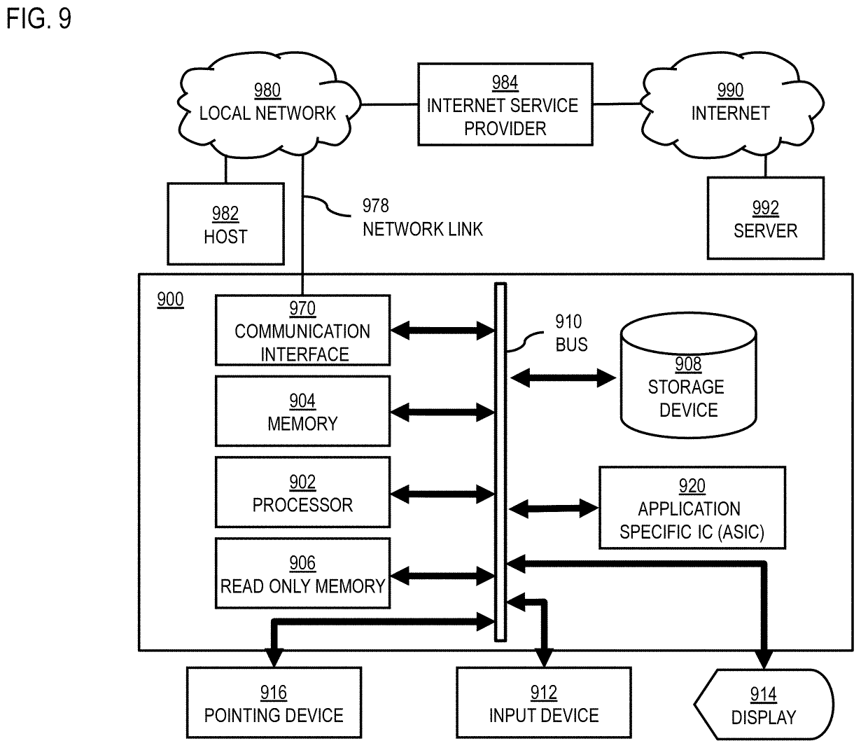

FIG. 9 illustrates a computer system 900 upon which an embodiment of the invention may be implemented. Computer system 900 is programmed (e.g., via computer program code or instructions) to provide lane-level mapping/routing based on vehicle/user health status data as described herein and includes a communication mechanism such as a bus 910 for passing information between other internal and external components of the computer system 900. Information (also called data) is represented as a physical expression of a measurable phenomenon, typically electric voltages, but including, in other embodiments, such phenomena as magnetic, electromagnetic, pressure, chemical, biological, molecular, atomic, sub-atomic and quantum interactions. For example, north and south magnetic fields, or a zero and non-zero electric voltage, represent two states (0, 1) of a binary digit (bit). Other phenomena can represent digits of a higher base. A superposition of multiple simultaneous quantum states before measurement represents a quantum bit (qubit). A sequence of one or more digits constitutes digital data that is used to represent a number or code for a character. In some embodiments, information called analog data is represented by a near continuum of measurable values within a particular range.

A bus 910 includes one or more parallel conductors of information so that information is transferred quickly among devices coupled to the bus 910. One or more processors 902 for processing information are coupled with the bus 910.

A processor 902 performs a set of operations on information as specified by computer program code related to providing lane-level mapping/routing based on vehicle/user health status data. The computer program code is a set of instructions or statements providing instructions for the operation of the processor and/or the computer system to perform specified functions. The code, for example, may be written in a computer programming language that is compiled into a native instruction set of the processor. The code may also be written directly using the native instruction set (e.g., machine language). The set of operations include bringing information in from the bus 910 and placing information on the bus 910. The set of operations also typically include comparing two or more units of information, shifting positions of units of information, and combining two or more units of information, such as by addition or multiplication or logical operations like OR, exclusive OR (XOR), and AND. Each operation of the set of operations that can be performed by the processor is represented to the processor by information called instructions, such as an operation code of one or more digits. A sequence of operations to be executed by the processor 902, such as a sequence of operation codes, constitute processor instructions, also called computer system instructions or, simply, computer instructions. Processors may be implemented as mechanical, electrical, magnetic, optical, chemical or quantum components, among others, alone or in combination.

Computer system 900 also includes a memory 904 coupled to bus 910. The memory 904, such as a random access memory (RANI) or other dynamic storage device, stores information including processor instructions for providing lane-level mapping/routing based on vehicle/user health status data. Dynamic memory allows information stored therein to be changed by the computer system 900. RANI allows a unit of information stored at a location called a memory address to be stored and retrieved independently of information at neighboring addresses. The memory 904 is also used by the processor 902 to store temporary values during execution of processor instructions. The computer system 900 also includes a read only memory (ROM) 906 or other static storage device coupled to the bus 910 for storing static information, including instructions, that is not changed by the computer system 900. Some memory is composed of volatile storage that loses the information stored thereon when power is lost. Also coupled to bus 910 is a non-volatile (persistent) storage device 908, such as a magnetic disk, optical disk, or flash card, for storing information, including instructions, that persists even when the computer system 900 is turned off or otherwise loses power.

Information, including instructions for providing lane-level mapping/routing based on vehicle/user health status data, is provided to the bus 910 for use by the processor from an external input device 912, such as a keyboard containing alphanumeric keys operated by a human user, or a sensor. A sensor detects conditions in its vicinity and transforms those detections into physical expression compatible with the measurable phenomenon used to represent information in computer system 900. Other external devices coupled to bus 910, used primarily for interacting with humans, include a display device 914, such as a cathode ray tube (CRT) or a liquid crystal display (LCD), or plasma screen or printer for presenting text or images, and a pointing device 916, such as a mouse or a trackball or cursor direction keys, or motion sensor, for controlling a position of a small cursor image presented on the display 914 and issuing commands associated with graphical elements presented on the display 914. In some embodiments, for example, in embodiments in which the computer system 900 performs all functions automatically without human input, one or more of external input device 912, display device 914 and pointing device 916 is omitted.

In the illustrated embodiment, special purpose hardware, such as an application specific integrated circuit (ASIC) 920, is coupled to bus 910. The special purpose hardware is configured to perform operations not performed by processor 902 quickly enough for special purposes. Examples of application specific ICs include graphics accelerator cards for generating images for display 914, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 900 also includes one or more instances of a communications interface 970 coupled to bus 910. Communication interface 970 provides a one-way or two-way communication coupling to a variety of external devices that operate with their own processors, such as printers, scanners, and external disks. In general the coupling is with a network link 978 that is connected to a local network 980 to which a variety of external devices with their own processors are connected. For example, communication interface 970 may be a parallel port or a serial port or a universal serial bus (USB) port on a personal computer. In some embodiments, communications interface 970 is an integrated services digital network (ISDN) card or a digital subscriber line (DSL) card or a telephone modem that provides an information communication connection to a corresponding type of telephone line. In some embodiments, a communication interface 970 is a cable modem that converts signals on bus 910 into signals for a communication connection over a coaxial cable or into optical signals for a communication connection over a fiber optic cable. As another example, communications interface 970 may be a local area network (LAN) card to provide a data communication connection to a compatible LAN, such as Ethernet. Wireless links may also be implemented. For wireless links, the communications interface 970 sends or receives or both sends and receives electrical, acoustic, or electromagnetic signals, including infrared and optical signals, that carry information streams, such as digital data. For example, in wireless handheld devices, such as mobile telephones like cell phones, the communications interface 970 includes a radio band electromagnetic transmitter and receiver called a radio transceiver. In certain embodiments, the communications interface 970 enables connection to the communication network 107 for providing lane-level mapping/routing based on vehicle/user health status data to the vehicle 101 and/or the UE 109.

The term computer-readable medium is used herein to refer to any medium that participates in providing information to processor 902, including instructions for execution. Such a medium may take many forms, including, but not limited to, non-volatile media, volatile media, and transmission media. Non-volatile media include, for example, optical or magnetic disks, such as storage device 908. Volatile media include, for example, dynamic memory 904. Transmission media include, for example, coaxial cables, copper wire, fiber optic cables, and carrier waves that travel through space without wires or cables, such as acoustic waves and electromagnetic waves, including radio, optical and infrared waves. Signals include man-made transient variations in amplitude, frequency, phase, polarization, or other physical properties transmitted through the transmission media. Common forms of computer-readable media include, for example, a floppy disk, a flexible disk, hard disk, magnetic tape, any other magnetic medium, a CD-ROM, CDRW, DVD, any other optical medium, punch cards, paper tape, optical mark sheets, any other physical medium with patterns of holes or other optically recognizable indicia, a RAM, a PROM, an EPROM, a FLASH-EPROM, any other memory chip or cartridge, a carrier wave, or any other medium from which a computer can read.

Network link 978 typically provides information communication using transmission media through one or more networks to other devices that use or process the information. For example, network link 978 may provide a connection through local network 980 to a host computer 982 or to equipment 984 operated by an Internet Service Provider (ISP). ISP equipment 984 in turn provides data communication services through the public, world-wide packet-switching communication network of networks now commonly referred to as the Internet 990.

A computer called a server host 992 connected to the Internet hosts a process that provides a service in response to information received over the Internet. For example, server host 992 hosts a process that provides information representing video data for presentation at display 914. It is contemplated that the components of system can be deployed in various configurations within other computer systems, e.g., host 982 and server 992.

FIG. 10 illustrates a chip set 1000 upon which an embodiment of the invention may be implemented. Chip set 1000 is programmed to provide lane-level mapping/routing based on vehicle/user health status data as described herein and includes, for instance, the processor and memory components described with respect to FIG. 10 incorporated in one or more physical packages (e.g., chips). By way of example, a physical package includes an arrangement of one or more materials, components, and/or wires on a structural assembly (e.g., a baseboard) to provide one or more characteristics such as physical strength, conservation of size, and/or limitation of electrical interaction. It is contemplated that in certain embodiments the chip set can be implemented in a single chip.

In one embodiment, the chip set 1000 includes a communication mechanism such as a bus 1001 for passing information among the components of the chip set 1000. A processor 1003 has connectivity to the bus 1001 to execute instructions and process information stored in, for example, a memory 1005. The processor 1003 may include one or more processing cores with each core configured to perform independently. A multi-core processor enables multiprocessing within a single physical package. Examples of a multi-core processor include two, four, eight, or greater numbers of processing cores. Alternatively or in addition, the processor 1003 may include one or more microprocessors configured in tandem via the bus 1001 to enable independent execution of instructions, pipelining, and multithreading. The processor 1003 may also be accompanied with one or more specialized components to perform certain processing functions and tasks such as one or more digital signal processors (DSP) 1007, or one or more application-specific integrated circuits (ASIC) 1009. A DSP 1007 typically is configured to process real-world signals (e.g., sound) in real time independently of the processor 1003. Similarly, an ASIC 1009 can be configured to performed specialized functions not easily performed by a general purposed processor. Other specialized components to aid in performing the inventive functions described herein include one or more field programmable gate arrays (FPGA) (not shown), one or more controllers (not shown), or one or more other special-purpose computer chips.

The processor 1003 and accompanying components have connectivity to the memory 1005 via the bus 1001. The memory 1005 includes both dynamic memory (e.g., RAM, magnetic disk, writable optical disk, etc.) and static memory (e.g., ROM, CD-ROM, etc.) for storing executable instructions that when executed perform the inventive steps described herein to provide lane-level mapping/routing based on vehicle/user health status data. The memory 1005 also stores the data associated with or generated by the execution of the inventive steps.

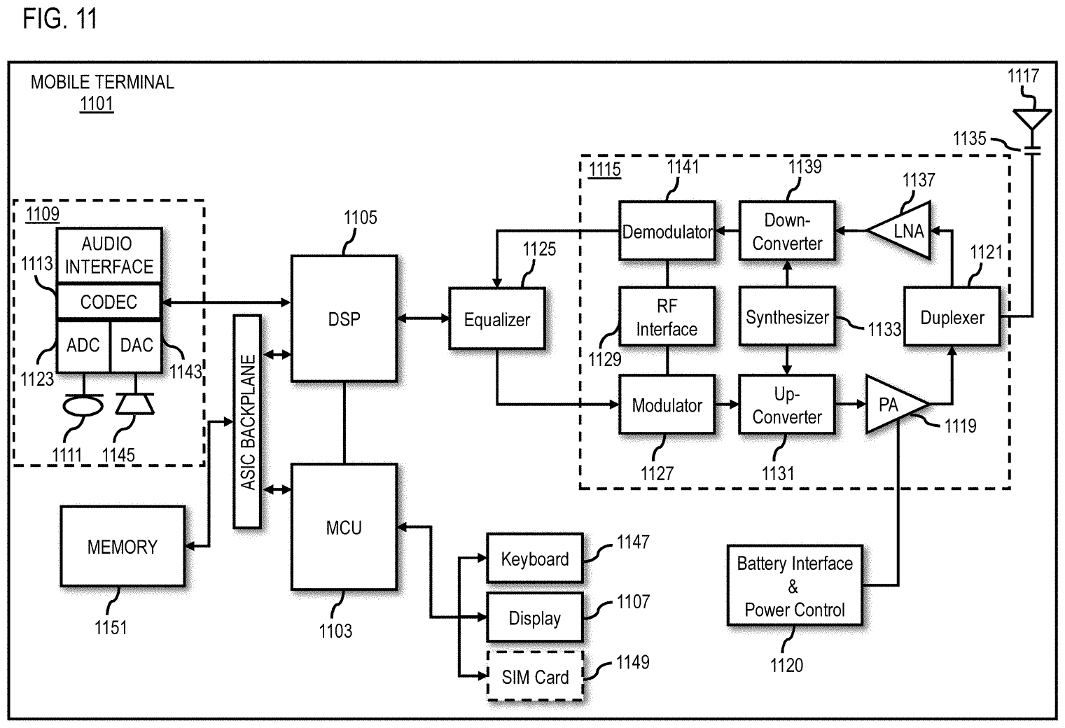

FIG. 11 is a diagram of exemplary components of a mobile terminal 1101 (e.g., handset, vehicle, or a part thereof) capable of operating in the system of FIG. 1, according to one embodiment. Generally, a radio receiver is often defined in terms of front-end and back-end characteristics. The front-end of the receiver encompasses all of the Radio Frequency (RF) circuitry whereas the back-end encompasses all of the base-band processing circuitry. Pertinent internal components of the telephone include a Main Control Unit (MCU) 1103, a Digital Signal Processor (DSP) 1105, and a receiver/transmitter unit including a microphone gain control unit and a speaker gain control unit. A main display unit 1107 provides a display to the user in support of various applications and mobile station functions that offer automatic contact matching. An audio function circuitry 1109 includes a microphone 1111 and microphone amplifier that amplifies the speech signal output from the microphone 1111. The amplified speech signal output from the microphone 1111 is fed to a coder/decoder (CODEC) 1113.

A radio section 1115 amplifies power and converts frequency in order to communicate with a base station, which is included in a mobile communication system, via antenna 1117. The power amplifier (PA) 1119 and the transmitter/modulation circuitry are operationally responsive to the MCU 1103, with an output from the PA 1119 coupled to the duplexer 1121 or circulator or antenna switch, as known in the art. The PA 1119 also couples to a battery interface and power control unit 1120.

In use, a user of mobile station 1101 speaks into the microphone 1111 and his or her voice along with any detected background noise is converted into an analog voltage. The analog voltage is then converted into a digital signal through the Analog to Digital Converter (ADC) 1123. The control unit 1103 routes the digital signal into the DSP 1105 for processing therein, such as speech encoding, channel encoding, encrypting, and interleaving. In one embodiment, the processed voice signals are encoded, by units not separately shown, using a cellular transmission protocol such as global evolution (EDGE), general packet radio service (GPRS), global system for mobile communications (GSM), Internet protocol multimedia subsystem (IMS), universal mobile telecommunications system (UNITS), etc., as well as any other suitable wireless medium, e.g., microwave access (WiMAX), Long Term Evolution (LTE) networks, code division multiple access (CDMA), wireless fidelity (WiFi), satellite, and the like.

The encoded signals are then routed to an equalizer 1125 for compensation of any frequency-dependent impairments that occur during transmission though the air such as phase and amplitude distortion. After equalizing the bit stream, the modulator 1127 combines the signal with a RF signal generated in the RF interface 1129. The modulator 1127 generates a sine wave by way of frequency or phase modulation. In order to prepare the signal for transmission, an up-converter 1131 combines the sine wave output from the modulator 1127 with another sine wave generated by a synthesizer 1133 to achieve the desired frequency of transmission. The signal is then sent through a PA 1119 to increase the signal to an appropriate power level. In practical systems, the PA 1119 acts as a variable gain amplifier whose gain is controlled by the DSP 1105 from information received from a network base station. The signal is then filtered within the duplexer 1121 and optionally sent to an antenna coupler 1135 to match impedances to provide maximum power transfer. Finally, the signal is transmitted via antenna 1117 to a local base station. An automatic gain control (AGC) can be supplied to control the gain of the final stages of the receiver. The signals may be forwarded from there to a remote telephone which may be another cellular telephone, other mobile phone or a land-line connected to a Public Switched Telephone Network (PSTN), or other telephony networks.

Voice signals transmitted to the mobile station 1101 are received via antenna 1117 and immediately amplified by a low noise amplifier (LNA) 1137. A down-converter 1139 lowers the carrier frequency while the demodulator 1141 strips away the RF leaving only a digital bit stream. The signal then goes through the equalizer 1125 and is processed by the DSP 1105. A Digital to Analog Converter (DAC) 1143 converts the signal and the resulting output is transmitted to the user through the speaker 1145, all under control of a Main Control Unit (MCU) 1103—which can be implemented as a Central Processing Unit (CPU) (not shown).

The MCU 1103 receives various signals including input signals from the keyboard 1147. The keyboard 1147 and/or the MCU 1103 in combination with other user input components (e.g., the microphone 1111) comprise a user interface circuitry for managing user input. The MCU 1103 runs a user interface software to facilitate user control of at least some functions of the mobile station 1101 to provide lane-level mapping/routing based on vehicle/user health status data. The MCU 1103 also delivers a display command and a switch command to the display 1107 and to the speech output switching controller, respectively. Further, the MCU 1103 exchanges information with the DSP 1105 and can access an optionally incorporated SIM card 1149 and a memory 1151. In addition, the MCU 1103 executes various control functions required of the station. The DSP 1105 may, depending upon the implementation, perform any of a variety of conventional digital processing functions on the voice signals. Additionally, DSP 1105 determines the background noise level of the local environment from the signals detected by microphone 1111 and sets the gain of microphone 1111 to a level selected to compensate for the natural tendency of the user of the mobile station 1101.

The CODEC 1113 includes the ADC 1123 and DAC 1143. The memory 1151 stores various data including call incoming tone data and is capable of storing other data including music data received via, e.g., the global Internet. The software module could reside in RAM memory, flash memory, registers, or any other form of writable computer-readable storage medium known in the art including non-transitory computer-readable storage medium. For example, the memory device 1151 may be, but not limited to, a single memory, CD, DVD, ROM, RAM, EEPROM, optical storage, or any other non-volatile or non-transitory storage medium capable of storing digital data.

An optionally incorporated SIM card 1149 carries, for instance, important information, such as the cellular phone number, the carrier supplying service, subscription details, and security information. The SIM card 1149 serves primarily to identify the mobile station 1101 on a radio network. The card 1149 also contains a memory for storing a personal telephone number registry, text messages, and user specific mobile station settings.

While the invention has been described in connection with a number of embodiments and implementations, the invention is not so limited but covers various obvious modifications and equivalent arrangements, which fall within the purview of the appended claims. Although features of the invention are expressed in certain combinations among the claims, it is contemplated that these features can be arranged in any combination and order.

What is claimed is:

1. A method comprising:
determining health status data indicating at least one health or maintenance condition of a vehicle, a user of the vehicle, or a combination thereof;
computing a lane-level navigation routing for the vehicle, another vehicle, or a combination thereof based on the health status data; and
providing the lane-level navigation routing as an output.

2. The method of claim 1, wherein the lane-level navigation routing provides guidance on which lane of a multi-lane road segment that the vehicle, the another vehicle, or a combination thereof should drive based on the health status data.

3. The method of claim 1, wherein the lane-level navigation routing is computed by a routing engine using a cost function to minimize a safety risk indicated by the health status data, to optimize traffic associated with the health status data, or a combination thereof.

4. The method of claim 1, further comprising:
processing vehicle location history data of the vehicle to determine a vehicle service date, a distance driven by the vehicle since a service visit, or a combination thereof,
wherein the health status data is determined based on the vehicle service date, the distance driven, or a combination thereof.

5. The method of claim 4, wherein the processing comprises using a trained predictive machine learning model to predict the health status data using the vehicle service date, the distance driven, the vehicle location history data, or a combination thereof as an input.

6. The method of claim 4, wherein the location history data comprises location data collected from one or more sensors of the vehicle.

7. The method of claim 1, further comprising:
processing calendar data, user location history data, or a combination thereof associated with the user of the vehicle to determine a user sleep time, a user work time, a health check-up date of the user, a current health condition of the user, or a combination thereof,
wherein the health status data is based on the user sleep time, the user work time, the health check-up date, the current health condition, or a combination thereof.

8. The method of claim 7, wherein the processing comprises using a trained predictive machine learning model to predict the user sleep time, the user work time, the health check-up date, the current health condition, the calendar data, the user location history data, or a combination thereof as an input.

9. The method of claim 1, further comprising:
mapping the health status data to digital map data of a geographic database; and
providing the mapped health status data as an output in a mapping user interface.

10. The method of claim 1, wherein the lane-level navigation routing provides guidance on which lane to drive on for the other vehicle to avoid the vehicle based on the health status data.

11. An apparatus comprising:
at least one processor; and
at least one memory including computer program code for one or more programs,
the at least one memory and the computer program code configured to, with the at least one processor, cause the apparatus to perform at least the following,
aggregate health status data from a plurality of vehicles, a plurality of users of the plurality of vehicles, or a combination thereof, wherein the health status data indicates at least one health or maintenance condition of the plurality of vehicle, the plurality of users, or a combination thereof;
map-match the aggregated health status data based on location data associated with the plurality of vehicles, the plurality of users, or a combination thereof;
generate a health status map data layer based on the map-matched health status data; and
provide the health status map data layer as an output.

12. The apparatus of claim 11, wherein the health status map data layer is used to provide lane-level navigation routing.

13. The apparatus of claim 12, wherein the lane-level navigation routing is generated to reduce a safety risk, an accident risk, or a combination thereof.

14. The apparatus of claim 11, wherein the apparatus is further caused to:
compute a lane-level safety risk score for one or more lanes of a road segment based on the map-matched health status data,
wherein the health status map data layer further includes the lane-level safety risk score.

15. The apparatus of claim 11, wherein the health status data is predicted from historical location data associated with the plurality of vehicles, the plurality of users, or combination thereof using a machine learning model.

16. The apparatus of claim 11, wherein the apparatus is further caused to:
perform a predictive analysis based on the map-matched health status data to determine which of the at least one health or maintenance condition is predicted to occur in a lane or a road segment.

17. A non-transitory computer-readable storage medium, carrying one or more sequences of one or more instructions which, when executed by one or more processors, cause an apparatus to at least perform the following steps:
receiving health status data associated with a vehicle, a user of the vehicle, or a combination thereof traveling in a road network;
processing the health status data to predict a safety risk, an accident risk, or a combination thereof associated with the vehicle, the user, or a combination thereof at a lane level of the road network; and
providing data for broadcasting the predicted safety risk, the predicted accident risk, or a combination thereof via a communication channel.

18. The non-transitory computer-readable storage medium of claim 17, wherein the predicted safety risk, the predicted accident risk, or a combination thereof is used to compute a lane-level navigation routing within the road network.

19. The non-transitory computer-readable storage medium of claim 17, wherein the lane-level navigation routing comprises shifting to a different lane, taking an exit, taking an alternate road segment, or a combination thereof to reduce the predicted safety risk, the predicted accident risk, or a combination thereof.

20. The non-transitory computer-readable storage medium of claim 17, wherein the health status data is predicted from historical location data associated with the vehicle, the user, or combination thereof using a machine learning model.
